(19) United States
(12) Patent Application Publication
(10) Pub. No.: US 2024/0335314 A1
(43) Pub. Date: Oct. 10, 2024

(54) GARMENT FOR STABILIZING A HUMAN ANATOMICAL JOINT

(71) Applicant: Stoko Design, Inc., Vancouver (CA)

(72) Inventors: Andrew CAMPBELL, Squamish (CA); Joel MAERZ, Vancouver (CA); Ryan BAKKER, Vancouver (CA)

(21) Appl. No.: 18/621,322

(22) Filed: Mar. 29, 2024

Related U.S. Application Data
(60) Provisional application No. 63/494,746, filed on Apr. 6, 2023.

(51) Int. Cl. A61F 5/01

(52) U.S. Cl. CPC A61F 5/0123; A61F 2005/0132

(57) ABSTRACT

The present invention involves a brace garment arranged for enveloping at least in part a joint of a human user comprises an extensible matrix fabric, at least one low elasticity web portion having a zonal fabric different from the matrix fabric, and at least one corresponding tension member disposed to induce tension in the at least one low elasticity web portion. The one boundary between the low elasticity web portion and the matrix fabric is a continuously knitted boundary. The garment may comprise conduits knitted into the matrix fabric and the low elasticity web portion for guiding the at least one tension member. The path of the tension member spatially relates the at least one tension member to at least one natural ligament of the knee, at least one of the low elasticity web portions being disposed to apply pressure to a side of the knee.

### CROSS-REFERENCE TO RELATED APPLICATIONS

The present application claims priority under 35 USC 119 (c) to U.S. Provisional Patent Application Ser. No. 63/494,746, filed Apr. 6, 2023.

### FIELD OF THE INVENTION

This present invention relates to the medical field as exemplified by IPC class A61 and more particularly to apparatus and associated methods for stabilizing articulating joints of the human body, including devices adapted to facilitate walking. In one aspect, it relates to an apparatus for stabilizing the human knee and the operation of such an apparatus configured and arranged for treating damaged ligaments in the knee.

### DESCRIPTION OF RELATED ART

Orthopedic braces are used to stabilize joints between the limbs of the human anatomy in cases where the joints or the limbs articulating about them have sustained damage. Braces have been employed to stabilize knees, ankles, elbows and wrists in this way. The brace is applied to reduce strain on the injured limb or joint while permitting the limb or joint to still perform its function, thereby minimizing the risk of further damage.

Several knee brace products have been developed to more specifically protect the ligaments of the knee, including the anterior cruciate ligament (ACL), posterior cruciate ligament (PCL), and medial collateral ligament (MCL). These are the ligaments most often damaged in when knees are overstrained, particularly by individuals involved in strenuous sporting activity. The lateral collateral ligament (LCL) may also sometimes become strained. The brace products for protecting these ligaments vary greatly in technology, function, and efficacy. Some products are focused on providing mechanical encapsulation that still allows articulation of the joint. Such products tend to involve rather heavy hinge mechanisms and are generally bulky and not particularly aesthetic. At the other extreme there are several products that assume the form of a garment, different forms of such garments employing different technologies to produce compressive forces in the general vicinity of the knee in an attempt to stabilize it and protect the above ligaments. Some garments fall short in providing enough compression. Others provide adequate overall compression but do not direct the compression to key anatomical areas.

There thus remains a need for a garment capable of accurately directing adequate force to stabilize an injured articulating human anatomical joint. In sportswear garment construction, knitted fabrics are widely used due to their inherent mechanical stretch properties. In contrast to woven fabrics, which generally do not stretch, knitted fabrics expand easily. This inherent mechanical stretch property is necessary to create form fitted garments that conform to the skin, including for example T-shirts, tights, and underwear. In traditional sportswear construction, knitted fabrics are purchased in bolts or rolls. They are then cut into individual pattern pieces and the individual pattern pieces are joined by seams into a garment. Different types of fabrics, differing for example by material, by stretch (Young's Modulus), or by stitch, can be joined together by seams to achieve specific performance qualities in the garment as a whole. These performance qualities include, for example, compression, temperature regulation, and comfort. This traditional method of constructing garments does have shortcomings in respect of comfort, efficiency, or visual appearance, or because the desired properties are simply not achievable by sewing together individual pattern pieces. There remains a need for methods of garment construction that address these shortcomings.

### SUMMARY OF THE INVENTION

At In a first embodiment of a garment for stabilizing a human knee, the garment is arranged for enveloping at least in part a first knee of a first leg of a human user, the garment comprising a belt disposed to fit about the waist of the user, a plurality of inextensible web portions and a first tension member disposed longitudinally along a corresponding predetermined first curved three-dimensional spatial path within or on a matrix of a garment fabric along the first leg, the first predetermined path spatially relating the first tension member to at least one natural ligament of the first knee, at least one of the plurality of inextensible web portions being disposed by the first tension member to apply pressure to one of the medial and the lateral side of the first knee. The first tension member may have a proximal end and a lateral end, the proximal end of the first tension member anchored to the belt and the distal end of the first tension member disposed to be anchored by a triceps surae of the first leg of the user.

The distal end of the first tension member may disposed to be anchored by the triceps surae of the user via a loop of the tension member extending more than once around the first leg of the user, the loop disposed to encircle the first leg of the user below the triceps surae of the first leg of the user. In other embodiments, the distal end of the first tension member may be disposed to be anchored by the triceps surac of the first leg of the user via a loop of the tension member disposed to extend about the triceps surae of the first leg of the user on one of the medial and lateral side of the first leg of the user and the at least one of the plurality of inextensible web portions may be disposed to apply pressure to the other of the medial and the lateral side of the first knee of the user. The proximal end of the first tension member may be anchored to the belt via a tensioner. The tensioner may be an adjustable tensioner disposed and configured to allow the tension in the first tension member to be adjusted.

The first tension member may comprise a plurality of the inextensible web portions and tensionable fasteners. In other embodiments, the first tension member may comprise an inextensible cable extending along and within one or more hollow guides within or on the matrix of the garment fabric and on or within the matrix of the plurality of inextensible web portions. The cable may cross over itself on or in the at least one of the plurality of inextensible web portions disposed to apply pressure to one of the medial and the lateral side of the knee.

The garment may further comprise a tension regulator for maintaining a tension in the first tension member when the first knee of the first leg of the human user is bent. The tension regulator may comprise a pulley affixed to an inextensible web portion disposed between the first knee and the waist of the user proximate the first knee.

The garment may further comprise a second tension member disposed longitudinally along a corresponding second predetermined curved three-dimensional spatial path within or on a matrix of a garment fabric along the first leg of the user, the second predetermined path spatially relating the second tension member to at least one natural ligament of the second knee, at least one of the plurality of inextensible web portions being disposed by the second tension member to apply pressure to the other of the medial and the lateral side of the second knee. The second tension member may have a proximal end and a lateral end, the proximal end of the second tension member anchored to the belt and the distal end of the second tension member disposed to be anchored by a triceps surae of the first leg of the user. In other embodiments, the distal ends of both the first and the second tension members may terminate in a joint inextensible web portion surrounding the first leg below the triceps surae of the first leg.

The garment may be further arranged for enveloping at least in part a second knee of a corresponding second leg of the user, the garment comprising a third tension member disposed longitudinally along a corresponding third predetermined curved three-dimensional spatial path within or on a matrix of a garment fabric along the second leg of the user, the third predetermined path spatially relating the third tension member to at least one natural ligament of the second knee, at least one of the plurality of inextensible web portions being disposed by the third tension member to apply pressure to one of the medial and the lateral side of the second knec.

In a more general sense, the embodiments of the brace garment are arranged for enveloping at least in part a first joint of a first appendage of a human user, the garment comprising a belt disposed to fit about a body part of the user, a plurality of inextensible web portions, and a first tension member disposed longitudinally along a corresponding predetermined first curved three-dimensional spatial path within or on a matrix of a garment fabric along the first appendage, the first predetermined path spatially relating the first tension member to at least one natural ligament of the first joint, at least one of the plurality of inextensible web portions being disposed by the first tension member to apply pressure to one of a first and a second side of the first joint.

In an embodiment, there is provided a knitted brace garment configured for enveloping at least a first joint of a human user when the garment is worn by the user, the garment comprising: an elastically extensible matrix fabric; at least one low elasticity web portion having a first zonal fabric different from the matrix fabric; and at least one segments of at least one tension member disposed to induce tension in the at least one low elasticity web portion, wherein at least one boundary between the at least one low elasticity web portion and the matrix fabric is a continuously knitted boundary. The first joint may be one of an ankle, elbow, wrist, shoulder, hip, neck, spine, and one or more vertebrae of the human user. The at least one low elasticity web portion may include an anchor band disposed to anchor the brace garment to a body of the human user.

The at least one tension member may be disposed along a path that spatially relates the at least one tension member to at least one natural ligament of the knee of the user, wherein at least one low elasticity web portion is disposed to apply pressure to one of a lateral side and a medial side of the knee of a wearer for bracing the knee. In one embodiment, the brace garment may be configured for enveloping a hip joint of a corresponding leg of a human user when the garment is worn by the user.

The brace garment may comprise continuously knitted conduits for guiding the at least one tension member wherein the conduits are knitted into at least one of the matrix fabric and the at least one low elasticity web portion. At least one of the conduits may extend along a curved path arranged to circumnavigate in part a sensitive feature of a body of a user when the garment is worn by the user, for example the peroneal nerve of a leg of the user.

The brace garment may comprise conduits disposed on top of the matrix fabric and the low elasticity web portions to guide the at least one tension member. The at least one tension member may be a closed loop. The at least one tension member may be anchored to the triceps surae of the user by a loop at least in part about the triceps surae. The brace garment may comprise a tensioner on the garment arranged for adjusting a tension in the at least one tension member. The at least one tension member may comprise a plurality of strands; and the garment may comprise a tension evener arranged for evening tension among the plurality of strands. The garment may comprise a belt and the at least one tension member may have a proximal end and a distal end, the proximal end of the at least one tension member being anchored to the belt and the distal end of the at least one tension member being disposed to be anchored by a triceps surae.

At least two segments of the at least one tension member may be disposed to longitudinally extend along curved paths that traverse the at least one low elasticity web portion. The curved paths may curve in opposing directions to induce tension in the at least one low elasticity web portion. The at least two segments of the at least one tension member may be disposed to longitudinally extend along a curved paths through the matrix fabric proximate two opposing boundaries between the at least one low elasticity web portion and the elastically extensible matrix. The curved paths may curve in opposing directions to induce tension in the at least one low elasticity web portion. Two segments of the at least one tension member may extend along two corresponding curved paths traversing the at least one low elasticity web portion to induce tension between the two segments of the at least one tension member.

Each of the at least one low elasticity web portions may comprise at least one portion of the first zonal fabric knitted into the matrix fabric. The at least one low elasticity web portion may comprise at least one section of a second zonal fabric having a continuously knitted boundary with the at least one section of the first zonal fabric. The brace garment may comprise at least one low elasticity web portion disposed to partially envelop at least one limb of the user.

In a further aspect a method is provided for creating a brace garment for enveloping at least a first joint of a human user when the garment is worn by the user, the method comprising continuously knitting into an elastically extensible matrix fabric at least one low elasticity web portion having a zonal fabric different from the matrix fabric; and continuously knitting into the elastically extensible matrix fabric conduits disposed for receiving inextensible tension members, wherein the tension members when received in the conduits are disposed to induce tension in the at least one low elasticity web portion, and wherein at least one boundary between the at least one low elasticity web portion and the matrix fabric is a continuously knitted boundary. The first joint may be one of an ankle, elbow, wrist, shoulder, hip, neck, spine, and one or more vertebrae of the human user.

The zonal fabric may be continuously knitted from the same yarn as the matrix fabric. The zonal fabric may be continuously knitted from a plurality of strands of the same yarn as the matrix fabric. The matrix fabric may be knitted using a first stitch pattern and the zonal fabric is continuously knitted from the same yarn as the matrix fabric using a second stitch pattern different from the first stitch pattern. The zonal fabric may be continuously knitted from one or more different yarns from the yarn of the matrix fabric. At least one of the different yarns may be a melting yarn. The zonal fabric may be continuously knitted from the same yarn as the matrix fabric and from one or more yarns that differ from the yarn of the matrix fabric. The matrix fabric may be knitted using a first stitch pattern and the zonal fabric is continuously knitted using a second stitch pattern different from the first stitch pattern.

The conduits may be continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

The zonal fabric may be continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

### BRIEF DESCRIPTION OF DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a drawing showing the general structure of the human left knee with the patella and its adhesions deleted for the sake of clarity.

FIG. 2A and FIG. 2B are drawings showing different views of a first embodiment of a medial collateral ligament bracing garment for protecting the human knee.

FIG. 3A and FIG. 3B are drawings showing different views of an embodiment of a medial collateral ligament bracing garment for protecting the human knee in which a different lower inextensible web is employed.

FIG. 4A and FIG. 4B are drawings showing different views of an embodiment of a lateral collateral ligament bracing garment for protecting the human knee.

FIG. 5A and FIG. 5B are drawings showing different views of an embodiment of a joint lateral and medial collateral ligament bracing garment for protecting the human knee.

FIG. 6A and FIG. 6B are drawings showing different views of another embodiment of a joint lateral and medial collateral ligament bracing garment for protecting the human knee in which the upper and lower inextensible webs of each leg are combined into single webs.

FIG. 7A and FIG. 7B are drawings showing different views of an embodiment of a medial collateral ligament bracing garment for protecting the human knee in which cables are employed to exert pressure on the medial side of the knee.

FIG. 8A and FIG. 8B are drawings showing different views of an embodiment of a lateral collateral ligament bracing garment for protecting the human knee in which cables are employed to exert pressure on the lateral side of the knee.

FIG. 9A and FIG. 9B are drawings showing different views of an embodiment of a combined lateral and medial collateral ligament bracing garment for protecting the human knee in which cables are employed to exert pressure on the lateral and medial sides of the knee.

FIG. 10A, FIG. 10B and FIG. 10C are drawings of different embodiments of cable and guide systems for the bracing garments of FIG. 7A, FIG. 7B, FIG. 8A, FIG. 8B, FIG. 9A and FIG. 9B.

FIG. 11 is a drawing of a web portion of a ligament brace garment arranged to dispose a cable guide to cross over itself on the web.

FIG. 12 shows a modification to the embodiment of FIG. 8B.

FIG. 13A and FIG. 13B are drawings of two views of an embodiment of a knitted brace garment for protecting the human knee in which cables are employed to exert pressure on the exterior of the knee.

FIG. 14A, FIG. 14B, and FIG. 14C show three different embodiments of a low elasticity web portion of the garment of FIG. 13A and FIG. 13B.

FIG. 14D shows one embodiment of stitch patterns and the continuously knitted boundary between them for the fabrics in FIG. 14A.

FIG. 14E shows an embodiment of a knitted conduit for an inextensible tension member traversing a low elasticity web portion within the low elasticity web portion.

FIG. 15A, FIG. 15B, and FIG. 15C show three different embodiments of a low elasticity web portion.

FIG. 16A and FIG. 16B are drawings of two views of an embodiment of a knitted brace garment for protecting the human knee in which cables are employed to exert pressure on the exterior of the knee.

FIG. 17A and FIG. 17B are drawings of two views of an embodiment of a knitted brace garment for protecting the human hip joints in which cables are employed to exert medially directed pressure on the hip joints.

FIG. 18A and FIG. 18B are drawings of two views of an embodiment of a knitted brace garment for protecting the human knee in which cables are employed to exert pressure on the exterior of the knees and in which pressure is avoided on the peronial nerves of the legs.

FIG. 19 shows a flow chart of a method for creating a brace garment for enveloping at least a first joint of a human user when the garment is worn by the user.

FIG. 20A and FIG. 20B show a knitted shoulder brace garment employing a tensioned low elasticity web portion to stabilize a shoulder joint.

FIG. 21 shows a view of a lateral side of a knitted ankle brace garment employing tensioned low elasticity web portions to stabilize an ankle joint.

FIG. 22A and FIG. 22B show a knitted wrist brace garment employing a tensioned low elasticity web portion to stabilize a wrist joint.

FIG. 23A, FIG. 23B and FIG. 23C show a knitted elbow brace garment employing a tensioned low elasticity web portion to stabilize an elbow joint.

FIG. 24A and FIG. 24B show a knitted spinal brace garment employing a tensioned low elasticity web portion to stabilize joints between spinal vertebrae.

Corresponding reference characters indicate corresponding parts throughout the several views. Although the drawings represent embodiments of the present invention, the drawings are not necessarily to scale and certain features may be exaggerated in order to better illustrate and explain the present invention. The exemplification set out herein illustrates an embodiment of the invention, in one form, and such exemplifications are not to be construed as limiting the scope of the invention in any manner.

### DETAILED DESCRIPTION OF THE INVENTION

The embodiments disclosed below is/are not intended to be exhaustive or limit the invention to the precise form disclosed in the following detailed description. Rather, the embodiment is chosen and described so that others skilled in the art may utilize its teachings.

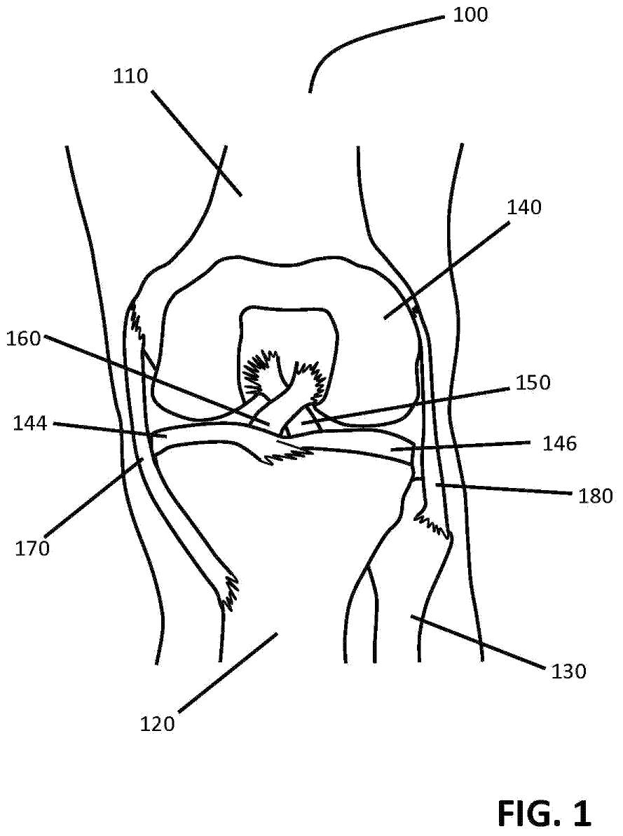

The present invention relates to a garment incorporating a brace arrangement for an articulating joint of a human body. In some embodiments, the brace comprises tensionable segments of inextensible material disposed to exert force on at least one natural ligament of the joint. In other embodiments, tension members disposed within conduits in segments of inextensible material provide the required force. In order to describe the garment and brace, we consider first the human knee as shown in FIG. 1.

FIG. 1 shows internal knee portion 100 of the human left leg as viewed from the front or anterior, but with the patella or knee cap and its various adhesions removed for the sake of clarity of the internal structure of this particular joint. The major bones of the leg are shown as femur 110, tibia 120 and fibula 130. The portions of these bones that have to contact one another during articulation are provided with articular cartilage 140, the two portions of the cartilage on tibia 120 being medial meniscus 144 and lateral meniscus 146. The bones are stabilized with respect to one another by strategically placed ligaments that hold them together. These include posterior cruciate ligament (PCL) 150 and anterior cruciate ligament (ACL) 160 binding together interacting faces of femur 110 and tibia 120, and medial collateral ligament (MCL) 170 and lateral collateral ligament (LCL) 180 binding respectively tibia 120 and fibula 130 to respectively the medial and lateral portions of the head of femur 110. The human right knee is substantially a mirror image of the left knee with respect to the medial plane.

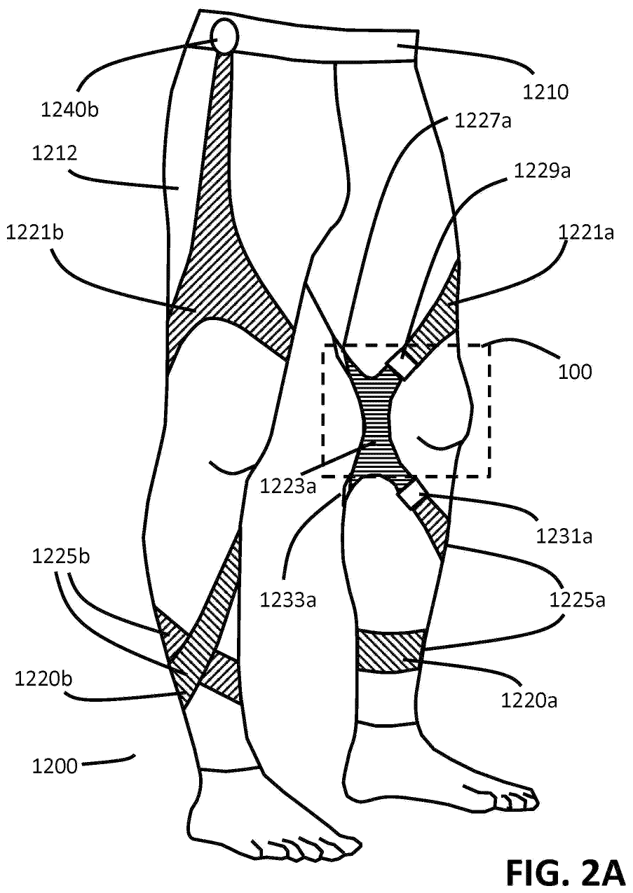

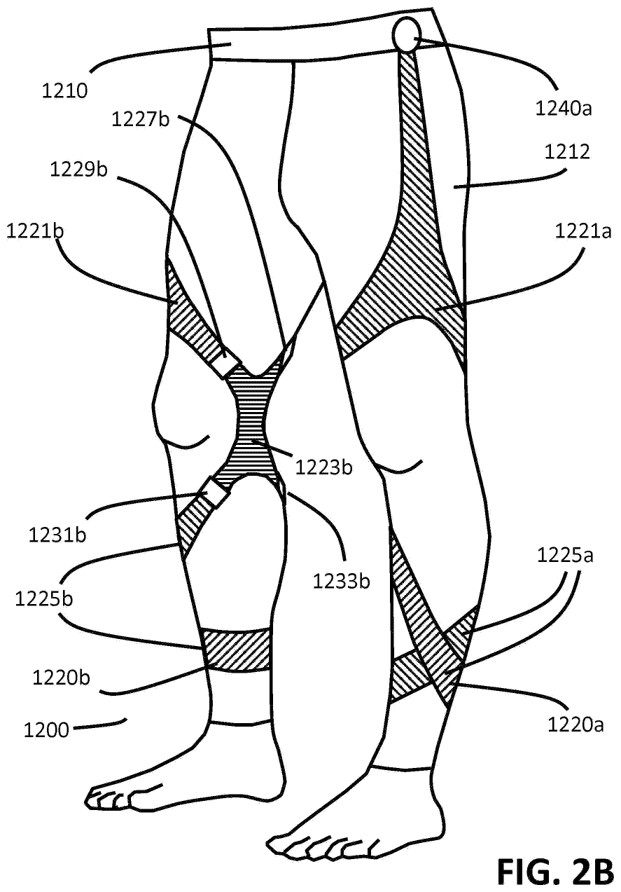

We turn now to a first embodiment of medial collateral ligament brace garment 1200, which may also be termed a bracing garment, shown in FIG. 2A and FIG. 2B, in which the knee joint of FIG. 1 is addressed by garment 1200 comprising belt 1210, extensible material 1212 forming the basic matrix material of garment 1200, tensioners 1240a and 1240b, and webs of substantially inextensible material 1221a, 1221b, 1225a, and 1225b.

The term “substantially inextensible material” is used herein to describe a material extending in two dimensions that is substantially inextensible in at least a first direction. The material may or may not have a restriction on extensibility in a direction perpendicular to the first direction. Non-limiting examples of such materials include but are not limited to so-called 2-way-stretch fabrics with blends of fabrics including Spandex, Nylon®, Dyneema™, Kevlar™, polyester, Ingeo™, olefin fibre, Lyocell, and/or cotton which are woven, knitted, or braided in such a fashion to allow stretch in required dimensions. The “2-way” stretching refers here to stretching in two opposing directions in a first dimension whilst remaining substantially inextensible in any direction perpendicular to the first direction. Other “substantially inextensible materials”, including for example without limitation fabrics comprising Dyneema™ fibers in a two-dimensional mesh embedded in a second fabric or in a sandwich structure, may have substantially no extensibility in any direction. The inextensibility is deemed “substantial” in comparison with the inextensibility of the matrix material of the fabric of the garment, the garment fabric being stretchable or extensible in comparison with the “substantially inextensible material”. We use herein the term “inextensible web” to describe a web made from a substantially inextensible material.

Broken rectangle 100 indicates the region of the left leg shown in FIG. 1. The webs of inextensible material form part of two independent tension members, being tension member 1220a for the left leg and tension member 1220b for the right leg, each joined to belt 1210. In order to reduce clutter in the drawings, each tension member is labeled only at its lower end in the drawings. Tension member 1220a comprises three webs of inextensible material, being upper inextensible web 1221a disposed on the left thigh, medial inextensible web 1223a disposed on the medial side of the left knee, and lower inextensible web 1225a disposed on the lower left leg, generally defining a curved three-dimensional spatial path which is spatially relating tension member 1220a to at least one ligament of joint 100. Tension member 1220a further comprises tensionable fasteners 1227a and 1229a attaching inextensible web 1221a to inextensible web 1223a. Tension member 1220a further comprises tensionable fasteners 1231a and 1233a attaching inextensible web 1223a to inextensible web 1225a. The same is correspondingly true of tension member 1220b, being comprised of corresponding inextensible webs 1221b, 1223b, and 1225b, as well as four corresponding fasteners, 1227a, 1229a, 1231b and 1233b, all obscured in FIG. 2A but visible in FIG. 2B.

Tension member 1220a may be joined to belt 1210 by means of adjustable tensioner 1240a (obscured in FIG. 2A, but visible in FIG. 2B) which is disposed and configured to allow the tension in tension member 1220b to be adjusted. Tension member 1220b may similarly be joined to belt 1210 by means of adjustable tensioner 1240a which is similarly disposed and configured to allow the tension in tension member 1220b to be adjusted.

Being directed to applying a suitable force to medial collateral ligament (MCL) 170, tension member 1220a is disposed to apply via inextensible web 1223a a force on the medial side of joint 100, the force on the medial side of the left knee being directed generally toward the lateral side of the left knee. This stabilizes the left knee joint against medial displacement as a result of problems with the medial collateral ligament of the left knee. Correspondingly, tension member 1220b is disposed to apply via inextensible web 1223b a force on the medial side of the right knee joint, the force on the medial side of the right knee being directed generally toward the lateral side of the right knee. This stabilizes the right knee joint against medial displacement as a result of problems with the medial collateral ligament of the right knee. The various tensionable fasteners may be implemented in a variety of well-known ways via, for example without limitation, ratchets, cam buckles, laces, straps with buckles, Velcro® fasteners, and the like.

FIG. 2A and FIG. 2B show the left and right knees both being stabilized by respective independent tension members 1220a and 1220b. In some embodiments there may be a tension member on only one of the two legs. Alternatively, there may be two tension members, one for each leg or knee, but only one is put under tension to apply to an affected or unstable knee. It will be understood by practitioners in the field that, if there is only a single tension member and it is applied to, for example, the left leg, then turning the garment inside out will render the tension member correctly arranged for application to the right leg to serve as stabilizing system for the right knee instead of the left knee.

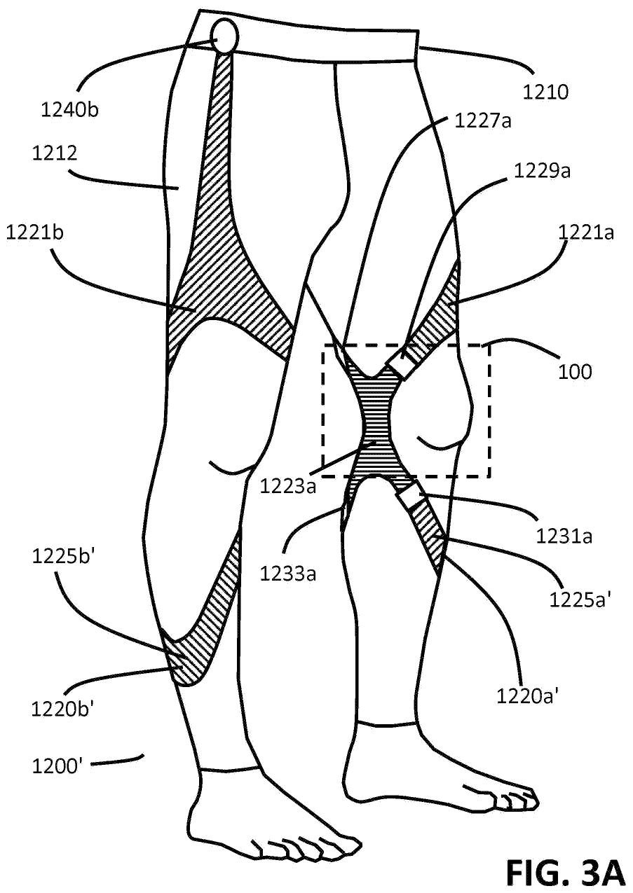

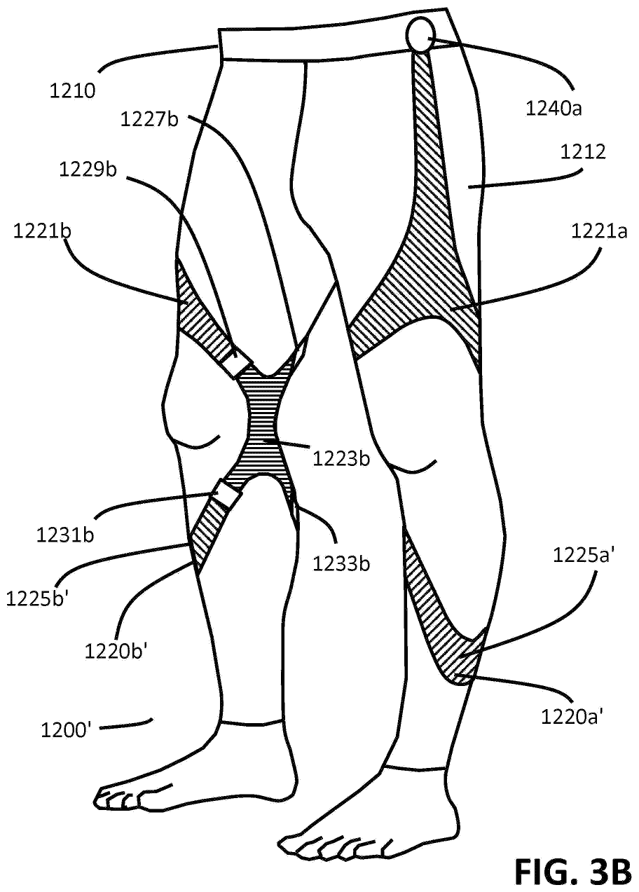

In FIG. 2A and FIG. 2B lower inextensible webs 1225a and 1225b are shown as looping around the leg below the triceps surae. In another embodiment of garment 1200′, shown in FIG. 3A and FIG. 3B, lower inextensible webs 1225a′ and 1225b′ do not loop around the leg, but simply extend partially around the leg below the triceps surae and effectively anchor themselves to the triceps surae. In order to reduce clutter in the drawings, each tension member is labeled only at its lower end in the drawings, being 1220a′ for the tension member on left leg and 1220b′ for the tension member on the right leg.

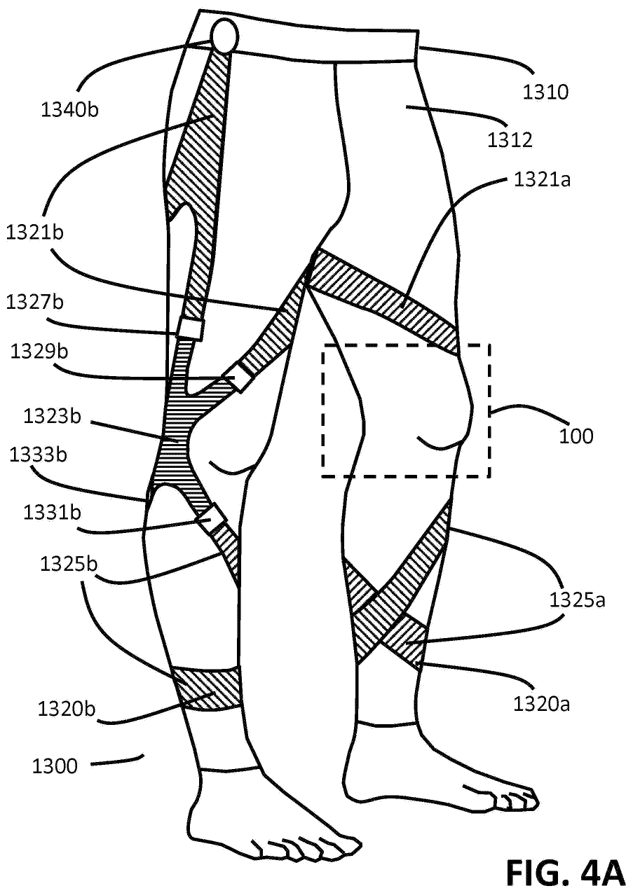

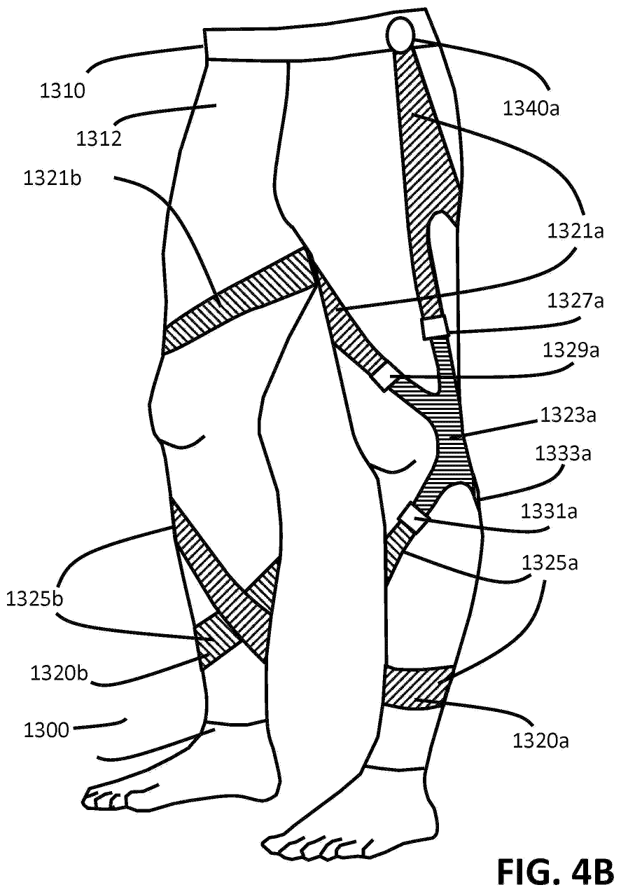

We turn now to a first embodiment of lateral collateral ligament brace garment 1300 shown in FIG. 4A and FIG. 4B, in which the knee joint of FIG. 1 is addressed by garment 1300 comprising belt 1310, extensible fabric 1312 forming the basic matrix material of garment 1300, webs of inextensible material, and tensioner 1340a (visible in FIG. 4B). Broken rectangle 100 indicates the region of the left leg shown in FIG. 1. The webs of inextensible material form part of two independent tension members, being tension member 1320a for the left leg and tension member 1320b for the right leg, each joined to belt 1310. Tension member 1320a comprises three webs of inextensible material, being upper inextensible web 1321a disposed on the left thigh and passing around it, lateral inextensible web 1323a disposed on the lateral side of the left knee, and lower inextensible web 1325a disposed on the lower left leg. Tension member 1320a further comprises tensionable fasteners 1327a and 1329a attaching inextensible web 1321a to inextensible web 1323a. Tension member 1320a further comprises tensionable fasteners 1331a and 1333a attaching inextensible web 1323a to inextensible web 1325a. The same is correspondingly true of tension member 1320b, being comprised of corresponding inextensible webs 1321b, 1323b, and 1325b, as well as four corresponding fasteners, 1327a, 1329a, 1331b and 1333b, all obscured in FIG. 4A but visible in FIG. 4B. In order to reduce clutter in the drawings, each tension member is labeled only at its lower end in the drawings, being 1320a for the tension member on left leg and 1320b for the tension member on the right leg.

Tension member 1320a may be joined to belt 1310 by means of adjustable tensioner 1340a (obscured in FIG. 4A, but visible in FIG. 4B) which is disposed and configured to allow the tension in tension member 1320b to be adjusted. Tension member 1320b may similarly be joined to belt 1310 by means of adjustable tensioner 1340a which is similarly disposed and configured to allow the tension in tension member 1320b to be adjusted.

Being directed to applying a suitable force to lateral collateral ligament (LCL) 180, tension member 1320a is disposed to apply via inextensible web 1323a a force on the lateral side of joint 100, the force on the lateral side of the left knee being directed generally toward the medial side of the left knee. This stabilizes the left knee joint against lateral displacement as result of problems with the lateral collateral ligament of the left kncc. Correspondingly, 1320b is disposed to apply via inextensible web 1323b a force on the lateral side of the right knee joint, the force on the lateral side of the right knee being directed generally toward the medial side of the right knee. This stabilizes the right knee joint against lateral displacement as result of problems with the lateral collateral ligament of the right knee. The various tensionable fasteners may be implemented in a variety of well-known ways via, for example without limitation, ratchets, cam buckles, laces, straps with buckles, Velcro® fasteners, and the like.

In FIG. 4A and FIG. 4B lower inextensible webs 1325a and 1325b are shown as looping around the leg below the triceps surae. As explained with reference to the medial collateral ligament brace garment of FIG. 2A and FIG. 2B, the lower inextensible webs may simply extend partially around the leg once below the triceps surae and effectively anchor themselves to the triceps surae, as for the medial collateral ligament brace system of FIG. 3A and FIG. 3B.

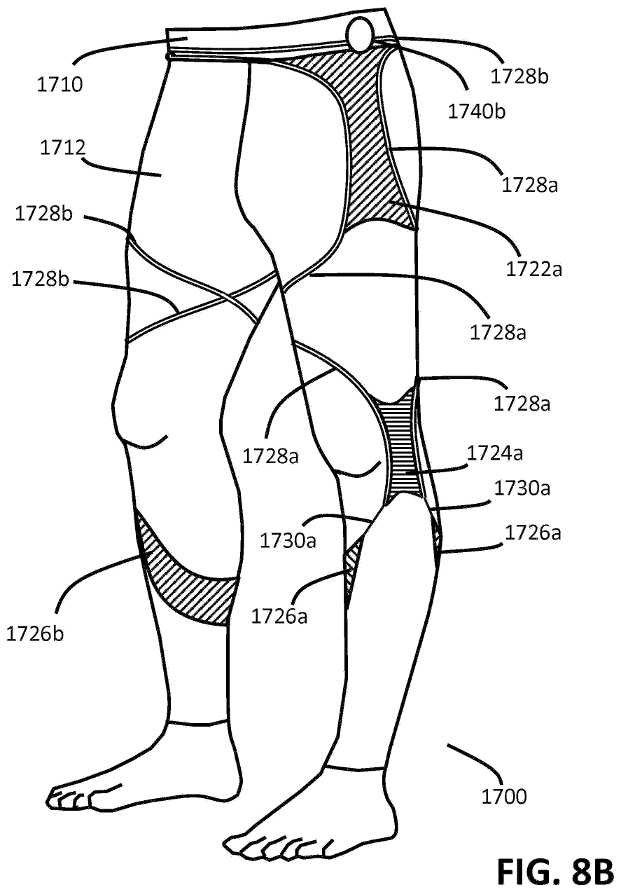

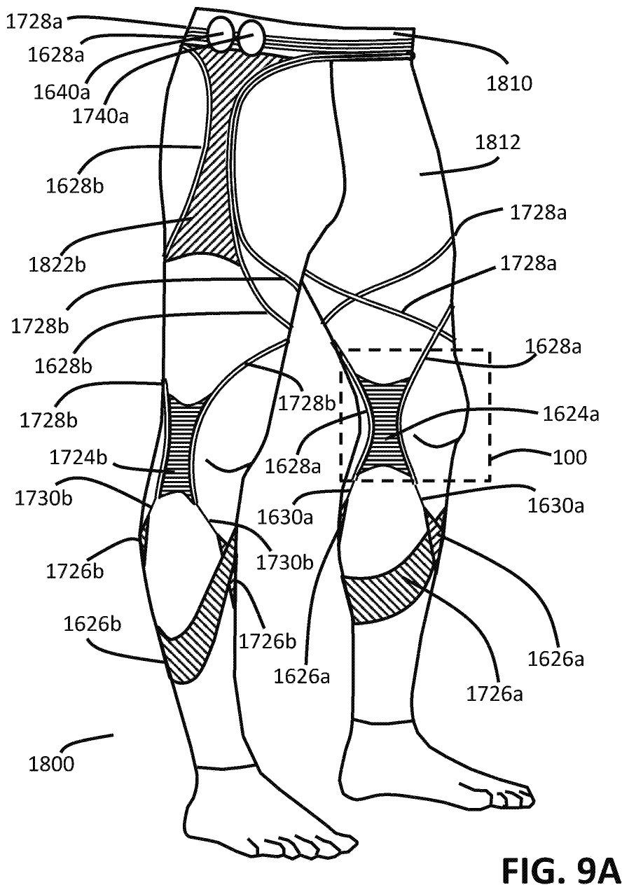

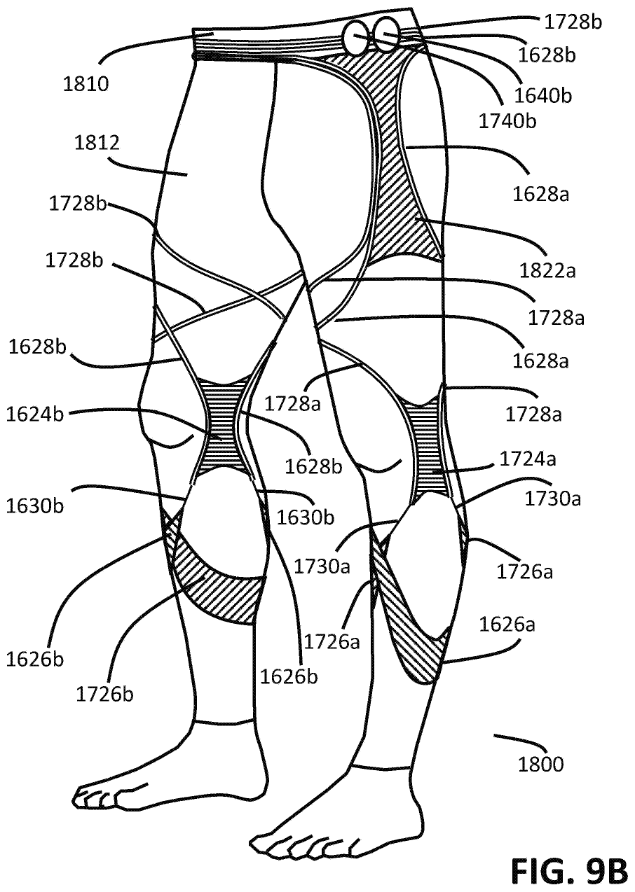

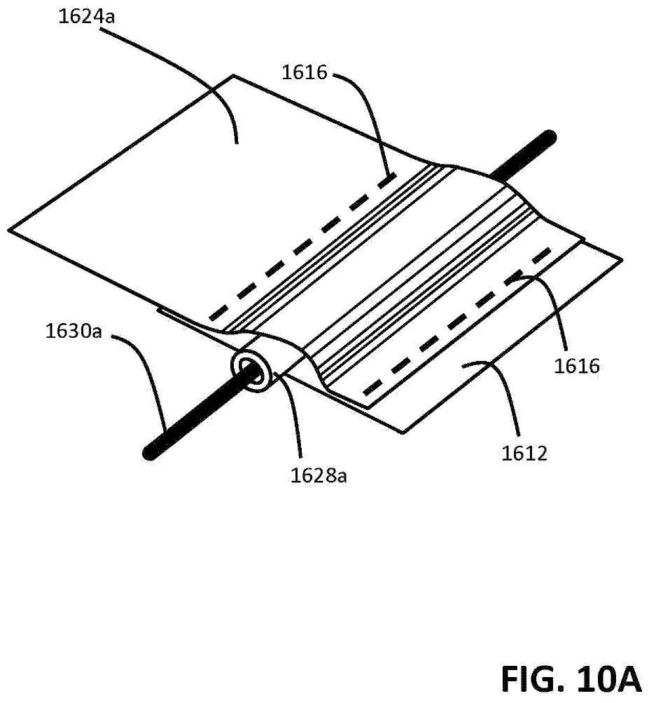

In FIG. 4A and FIG. 4B one upper limb of inextensible webs 1323a and 1323b are shown as connected directly to respectively web 1321a and 1321b via respectively fasteners 1327a and 1327b. In this implementation, the particular limbs of webs 1323a and 1323b therefore extend vertically along the thigh. In alternative embodiments, later used in FIG. 8A, FIG. 8B, FIG. 9A and FIG. 9B, web 1321a and 1321b may have extensions that extend around first the anterior, then the medial side, and then the posterior of the corresponding leg to be joined to webs 1323a and 1323b via fasteners 1327a and 1327b respectively. This particular arrangement may be better understood at the hand of FIG. 8A, FIG. 8B, FIG. 9A and FIG. 9B below.

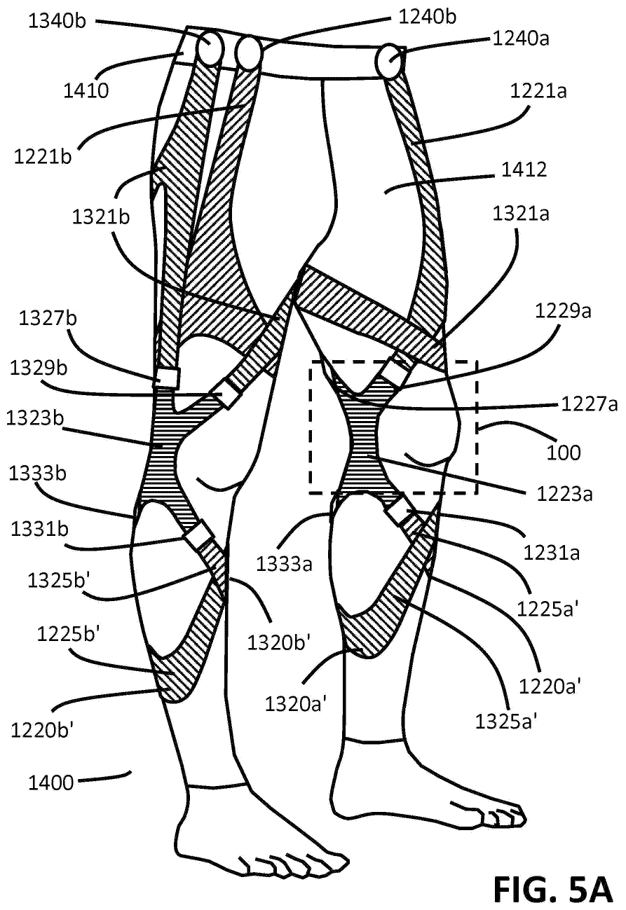

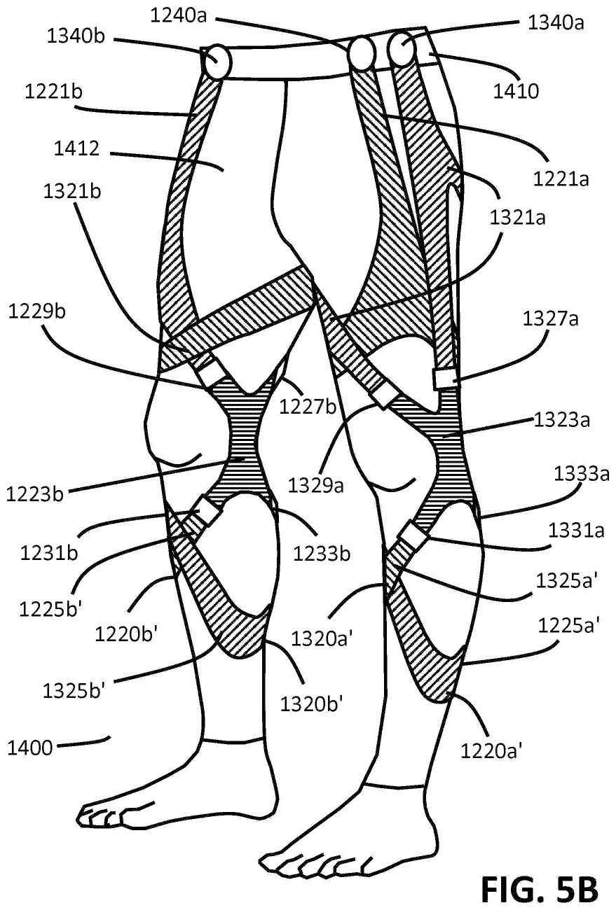

In yet a further embodiment, garment 1400 may have two tension members for a given leg with lateral and medial inextensible webs respectively acting on the lateral and medial sides of a particular knee. FIG. 5A and FIG. 5B show this implementation. The numbering follows the medial collateral ligament brace garment numbering of FIG. 3A and FIG. 3B and the lateral collateral ligament brace garment numbering of FIG. 4A and FIG. 4B, with the following exceptions. The belt is labeled 1410 and the extensible material or fabric of the garment is labeled 1412. The lower inextensible webs of the lateral collateral ligament brace garment numbering are given as 1325a′ for the left leg and 1325b′ for the right leg, since the lateral collateral ligament brace garment employs the same kind of lower inextensible web as the medial collateral ligament brace garment of FIG. 3A and FIG. 3B. The tensionable members bearing medial collateral ligament braces for the left and right leg are respectively labeled 1320a′ and 1320b′ proximate their lower ends. As may be seen in FIG. 5A and FIG. 5B, tensioners 1240a and 1240b are moved more toward the anterior of the body to provide space for upper inextensible webs 1221a and 1221b of the lateral collateral ligament brace garment arrangement and for their respective associated tensioners 1340a and 1340b. This embodiment is therefore a direct combination into a single garment of the lateral collateral ligament brace of FIG. 4A and FIG. 4B and the medial collateral ligament brace of FIG. 3A and FIG. 3B, with the exception that the lateral collateral ligament brace has been provided with the same kind of lower inextensible web as the medial collateral ligament brace of FIG. 3A and FIG. 3B.

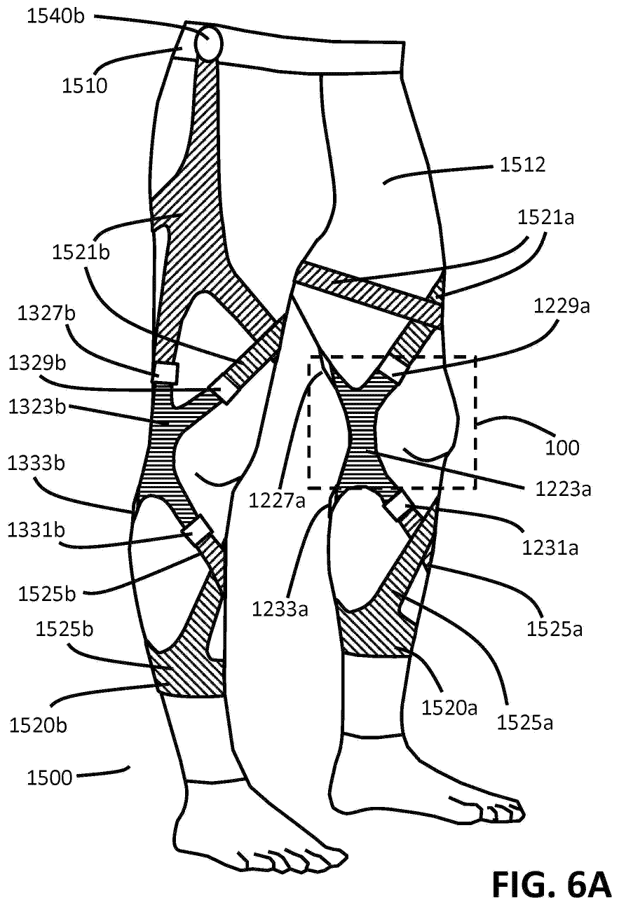

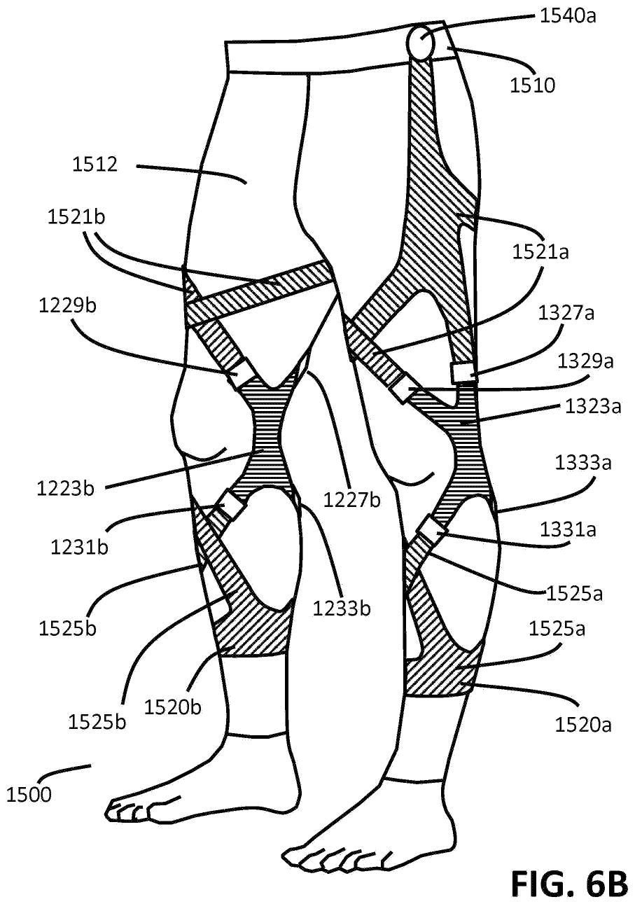

Based on the garment of FIG. 5A and FIG. 5B, the upper inextensible webs of the two braces may be combined for each leg into one upper inextensible web for each leg. Similarly, the lower inextensible webs of the two braces may be combined for each leg into one lower inextensible web for each leg. The result is garment 1500 of FIG. 6A and FIG. 6B. In this implementation, single lower inextensible web, 1525a for the left leg and 1525b for the right leg, may be connected to both the medial and lateral inextensible webs. Similarly, single upper inextensible web, being 1521a for the left leg and 1521b for the right leg, may be connected to both the medial and lateral inextensible webs. The garment has belt 1510, tensioners 1540a and 1540b. The material forming the basic matrix of the garment is labeled 1512. In this embodiment one leg of the garment has four inextensible webs, being one for the thigh, having four tensionable fasteners for connecting to the medial and lateral inextensible webs, one inextensible web on the medial side of the knee, one inextensible web on the lateral side of the knee, and a single lower inextensible web below the distal portion of the triceps surae. In this embodiment, the lower inextensible web has four tensionable fasteners, two for connecting to the medial inextensible web and two for connecting to the lateral inextensible web. In this embodiment each leg has a single tension member, being 1520a for the left leg and 1520b for the right leg.

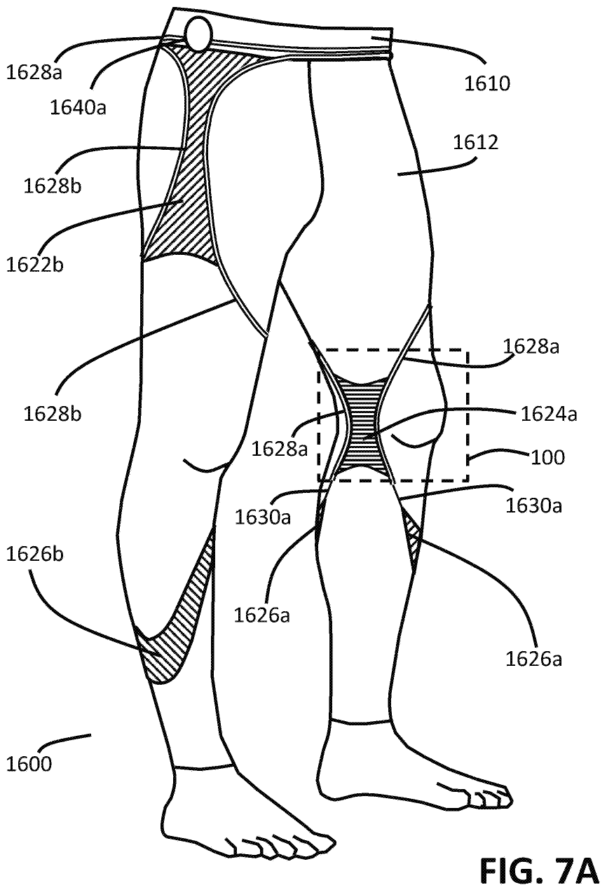

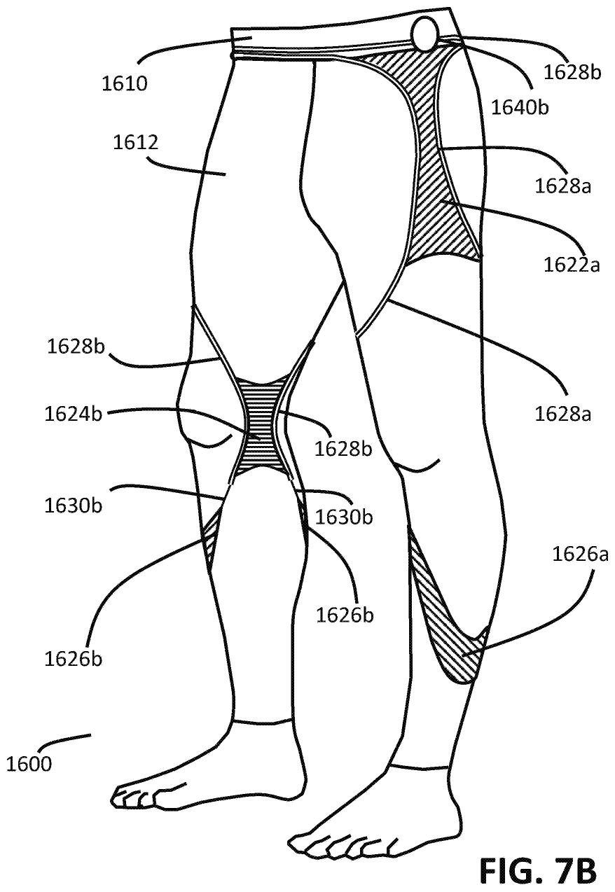

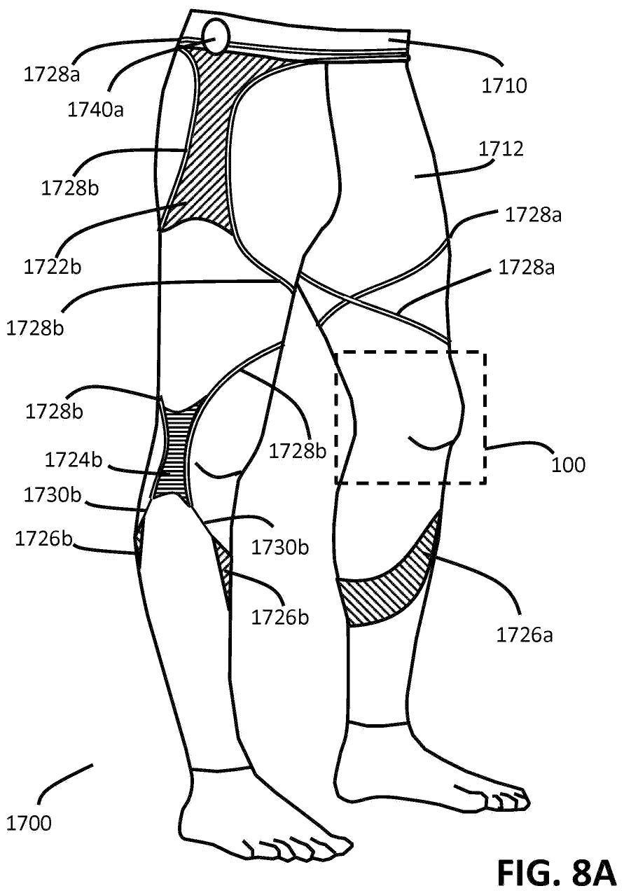

In another embodiment, shown in FIG. 7A and FIG. 7B, the knee joint of FIG. 1 is addressed by medial collateral ligament brace garment 1600 comprising belt 1610, tensioners 1640a for the left leg and 1640b for the right leg, and tension members for the left leg and for the right leg in the form of cables arranged to move in hollow guides within or on inextensible material webs. The cables may be made from a substantially inextensible material, for example without limitation, PTFE, stainless steel, Nylon®, Kevlar®, one or more ultra-high molecular weight polyethylene-based fiber, and a fiber with a diamond weave. The fiber with a diamond weave may be, for example without limitation, cotton, polyester, polypropylene, and Technora®. Broken rectangle 100 indicates the region of the left leg shown in FIG. 1. In this embodiment, tensioned cables are the means for generating the required force on the knee joint while the webs of inextensible material either serve as anchors to the torso, thigh or lower leg, or they translate to the knee joint the forces generated by the tension in the cables. In order to perform its function, the cable is substantially inextensible.

In FIG. 7A and FIG. 7B, inextensible cable 1630a is attached at its two ends to the two ends of lower inextensible web 1626a. Cable 1630a otherwise runs inside hollow guide 1628a. Inextensible web 1624a, which applies pressure to the medial side of the left leg, is attached at its two horizontal ends to hollow guide 1628a. When the tension in cable 1630a is increased, the pressure on the medial side of the left knee is commensurately increased. In one embodiment, hollow guide 1628a routes cable 1630a around the waist within belt 1610. Upper inextensible material web 1622a assures the positioning of guide segment1628a. Tensioner 1640a is disposed within the line of cable 1630a in order to adjust the tension in cable 1630a. In the embodiment shown in FIG. 7A and FIG. 7B, tensioner 1640a is disposed on the right hip.

Being directed to applying a suitable force to medial collateral ligament (MCL) 170, inextensible web 1624a is disposed to apply a force on the medial side of joint 100, the force being directed generally toward the lateral side of the left knee. This stabilizes the left knee joint against medial displacement as result of problems with the medial collateral ligament of the left knee. Correspondingly, inextensible web 1624b is disposed to apply a force on the medial side of the right knee, the force being directed generally toward the lateral side of the right knee. This stabilizes the right knee joint against medial displacement as result of problems with the medial collateral ligament of the right knee. To this end, cable 1630b is fastened at its two ends to the two ends of lower inextensible material web 1626b. Hollow guide 1628b routes cable 1630b around the waist within belt 1610. Upper inextensible material web 1622b assures the positioning of guide 1628b. Tensioner 1640b is disposed within the line of cable 1630b in order to adjust the tension in cable 1630a. In the embodiment shown in FIG. 7A and FIG. 7B, tensioner 1640b is disposed on the left hip.

In FIG. 8A and FIG. 8B, the knee joint of FIG. 1 is addressed by lateral collateral ligament brace garment 1700 comprising belt 1710, tensioners 1740a for the left leg and 1740b for the right leg, and tension members for the left leg and for the right leg in the form of cables arranged to move in hollow guides within or on inextensible material webs. Broken rectangle 100 indicates the region of the left leg shown in FIG. 1. Tensioned cables are the mechanism for generating the required force on the knee joint while the webs of inextensible material either serve as anchors to the torso, thigh or lower leg, or they translate to the knee joint the forces generated by the tension in the cables. In order to perform its function, the cable is substantially inextensible.

In FIG. 8A and FIG. 8B, cable 1730a is attached at its two ends to the two ends of lower inextensible web 1726a. Cable 1730a otherwise runs inside hollow guide 1728a on extensible material or matrix fabric 1712 of garment 1700. Inextensible web 1724a, which applies pressure to the lateral side of the left leg, is attached at its two horizontal ends to hollow guide 1728a. When the tension in cable 1730a is increased, the pressure on the lateral side of the left knee is commensurately increased. In one embodiment, hollow guide 1728a routes cable 1730a around the waist within belt 1710. Upper inextensible material web 1722a assures the positioning of guide 1728a. Tensioner 1740a is disposed within the line of cable 1730a in order to adjust the tension in cable 1730a. In the embodiment shown in FIG. 8A and FIG. 8B, tensioner 1740a is disposed on the right hip.

Being directed to applying a suitable force to lateral collateral ligament (LCL) 180, inextensible web 1724a is disposed to apply a force on the lateral side of joint 100, the force being directed generally toward the medial side of the left knee. This stabilizes the left knee joint against lateral displacement as result of problems with the lateral collateral ligament of the left knee. Correspondingly, inextensible web 1724b is disposed to apply a force on the lateral side of the right knee, the force being directed generally toward the medial side of the right knee. This stabilizes the right knee joint against lateral displacement as result of problems with the lateral collateral ligament of the right knee. To this end, cable 1730b is fastened at its two ends to the two ends of lower inextensible material web 1726b. Hollow guide 1728b routes cable 1730b around the waist within belt 1710. Upper inextensible material web 1722b assures the positioning of guide 1728b. Tensioner 1740b is disposed within the line of cable 1730b in order to adjust the tension in cable 1670a. In the embodiment shown in FIG. 8A and FIG. 8B, tensioner 1740b is disposed on the left hip.

In yet a further embodiment, garment 1800 may have two tension members for a given leg with lateral and medial inextensible webs respectively acting on the lateral and medial sides of a particular knee. FIG. 9A and FIG. 9B show this implementation. The numbering follows the medial collateral ligament brace garment numbering of FIG. 7A and FIG. 7B and the lateral collateral ligament brace garment numbering of FIG. 8A and FIG. 8B, with the following exceptions. The belt is labeled 1810, the extensible material or fabric of the garment is labeled 1812, and the upper inextensible web is labeled 1822a and 1822b for left and right legs respectively. As may be seen in FIG. 9A and FIG. 9B, tensioners 1640a, 1640b, 1740a and 1740b are relocated slightly from their positions in the earlier drawings in order to accommodate four of these devices. This embodiment is in its effect a direct combination into a single garment of the lateral collateral ligament brace of FIG. 8A and FIG. 8B and the medial collateral ligament brace of FIG. 7A and FIG. 7B, with the exception that the upper inextensible webs of the earlier diagrams have been combined into a single web. The two lower inextensible webs on each leg may be combined in the same arrangement as shown in FIG. 6A and FIG. 6B.

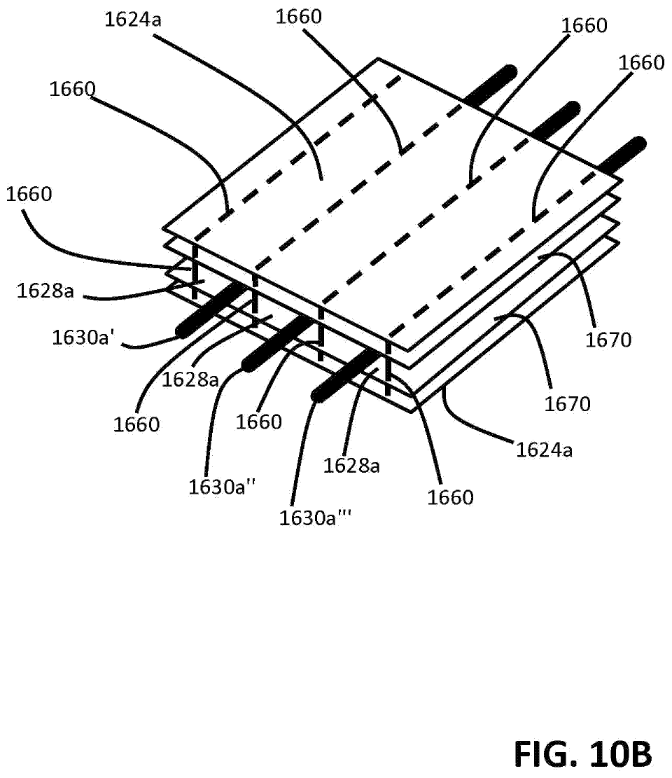

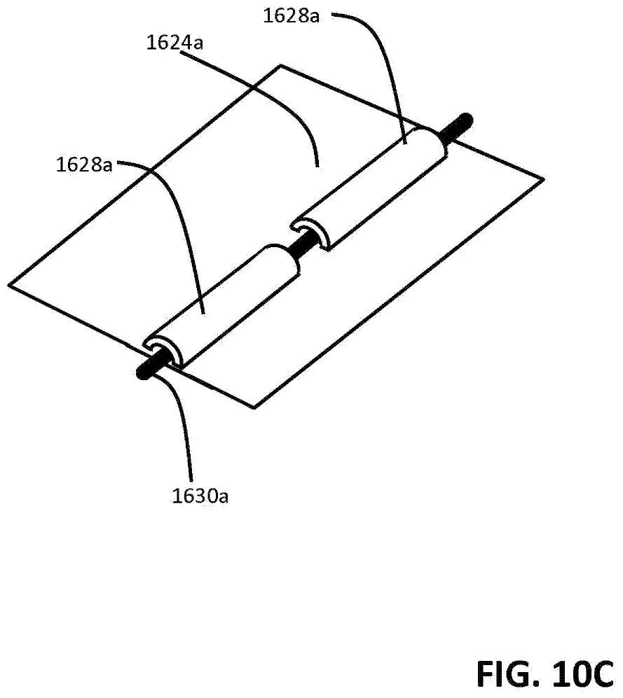

FIG. 10A, FIG. 10B, and FIG. 10C show different implementations of guides 1628a, 1628b, 1728a, and 1728b for use respectively with the cables 1630a, 1630b, 1730a, 1730b of FIG. 7A, FIG. 7b, FIG. 8A, FIG. 8B, FIG. 9A and FIG. 9B. Using the elements of FIG. 7A as example, FIG. 10A shows a seam at the joint created by stitches 1616 between matrix fabric 1612 of garment 1600 and medial collateral web 1624a, with guide 1628a extending along the seam in the form of tube 1628a. Cable 1630a extends longitudinally through tube 1628a. The material of tube 1628a is selected to have a low coefficient of friction with cable 1630a. In a more general case, there may be a plurality of joints, each having tube 1628a extending longitudinally along it and each tube 1628a having a strand of cable 1630a extending longitudinally along it. In FIG. 10A, tube 1628a is shown as circular in cross-section, but in other embodiments it may have any suitable cross-section that allows the strand of cable 1630a to move substantially freely, while simultaneously making tube 1628a compatible with the ergonomic requirements to which the garment is subject. One suitable cross-section is semi-circular, or a smaller segment of a circle with enough curvature to accommodate the strand of cable 1630a, yet be flat on one side so as to be easily integrated in matrix material 1612 of garment 1600. Tube 1628a may be collated, which allows very low friction, but inextensible tubing to be employed. Example materials for tubing 1628a include, but are not limited to Teflon® and silica. In a related embodiment, the strand of cable 1630a is simply sandwiched between two strips of low fiction material inside a stitched scam or joint. The same arrangements may be applied to the cables and guides of FIG. 7B, FIG. 8A, FIG. 8B, FIG. 9A and FIG. 9B.

FIG. 10B shows another embodiment of a way to incorporate cable 1630a of FIG. 7A within the inextensible material of medial collateral web 1624a of garment 1600. The drawing is an isometric view of the weave of the fabric of web 1624a. It shows four rows 1660 of stitching or weave either side of every one of three strands of cable 1630a within the matrix material of medial collateral web 1624a, but proximate the edge of web 1624a. Three strands of cable 1630a, being 1630a′, 1630a″, and 1630a′″, are sandwiched between two longitudinal strips 1670 of low friction material held by stitching 1660. In this drawing, the material of web 1624a is shown as being two sheets. This should be considered as presented schematically for the sake of clarity, as the material is in practice woven. The fiber of the stitching may be a low friction material so that cable 1630a slides on all sides against low friction material. In this embodiment, guides 1628a are formed by low friction material strips 1670 and low friction material stitching 1660. The same arrangements may be applied to the cables and guides of FIG. 7B, FIG. 8A, FIG. 8B, FIG. 9A and FIG. 9B.

FIG. 10C shows an embodiment in which guide 1628a is attached to the surface of garment 1600 in the form of collated semi-cylindrical tubes 1628a and cable 1630a routed through tubes 1628a. In some embodiments, the bases of the tubes may be closed or may be lined with a low friction material. The same arrangements may be applied to the cables and guides of FIG. 7B, FIG. 8A, FIG. 8B, FIG. 9A and FIG. 9B.

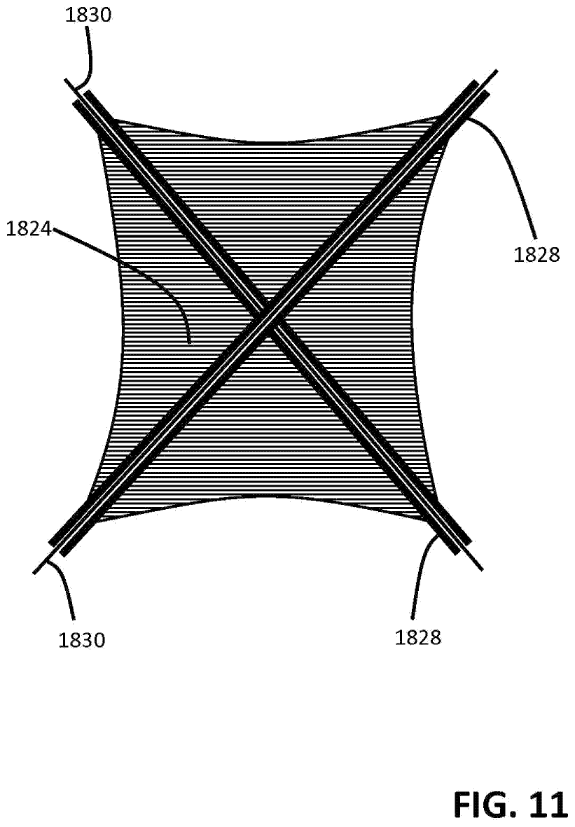

FIG. 7A, FIG. 7B, FIG. 8A, FIG. 8B, FIG. 9A and FIG. 9B all show guides 1628a, 1628b, 1728a, and 1728b as being disposed along the perimeters of webs 1624a, 1624b, 1724a, and 1724b. In an alternative embodiment, shown in FIG. 11, guide 1828 may be arranged to cross over itself on medial or lateral collateral web 1824 in order to guide corresponding cable 1830 between diagonally opposing corners of the web. This provides more stabilization of the posteriolateral corner and limits excessive internal and external rotation, thereby protecting the anterior cruciate ligament (ACL) 160 of FIG. 1. In FIG. 11 the guide arrangement of FIG. 10A is used. Any other guide arrangement that would cross cable 1830 over itself may be employed.

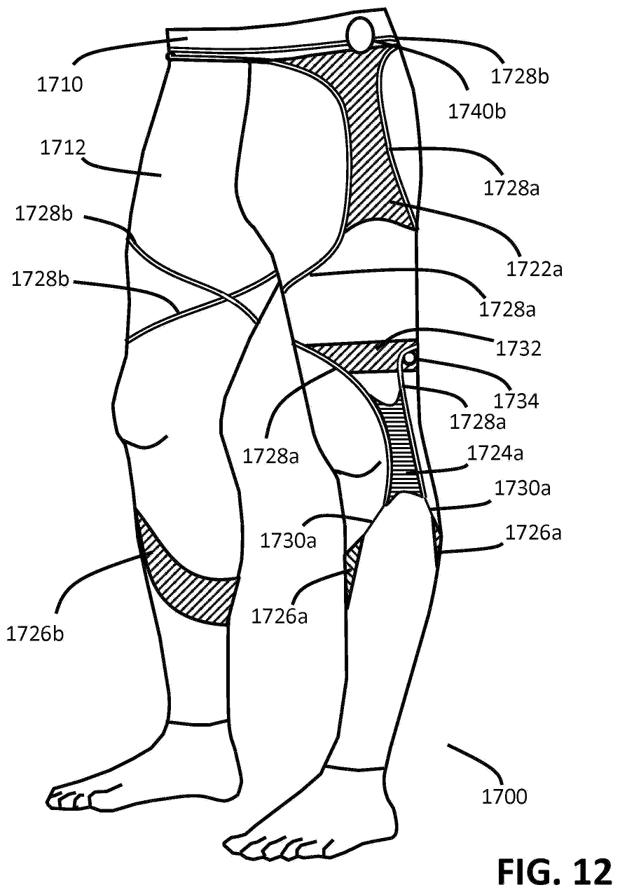

We turn now to FIG. 12, which shows lateral collateral brace garment of FIG. 8B to which a further inextensible web portion 1732 bearing pulley 1734 has been added, pulley 1734 affixed to inextensible web portion 1732 disposed between the knee and the waist of the user proximate the knee. In order to ensure that suitable tension is maintained in cable 1730a when the left leg is bent, guide 1728a is routed over pulley 1734, which serves as a tension regulator. Pulley 1734 represents just one implementation of a tension regulator by which tension may be maintained in cable 1730a when the leg is bent. Other tension regulators may also be employed, for example without limitation, the guide designs of FIG. 10A, FIG. 10B, and FIG. 10C. Tension regulators may similarly be employed to regulate tension in any of the other tension members disclosed herein.

With the more recent advent of computer-controlled knitting machines, fabrics may now also be produced with spatially varying properties. A suitable example industrial knitting machine with this capability is the STOLL CMS 530 HP manufactured by Karl Meyer Stoll of Reutlingen in Germany. Using a machine of this type, stitch patterns can be designed using a computer program loaded into the controller computer of the machine. These stitch patterns may be made with a chosen yarn, or plurality of yarns, of selected materials to achieve a desired fabric property. A continuously knitted combination of different segments of different types of fabrics may be produced in this way to achieve similar performance qualities as that of traditional cut and sew garments, but with fewer required seams. This may be done to increase comfort, efficiency, or visual appearance, or because the desired properties are unachievable using traditional cut and sew methods. Continuously knitted panels may be produced with segments seamlessly knitted together in one knitting process, the knitting process being continuous across any boundary between any two segments in a panel. These continuously knitted panels may then be joined by a very small number of seams to form the final garment. This stands in contrast to a prior art garment composed of many distinct panels of different fabrics joined together by way of seams between all adjacent panels.

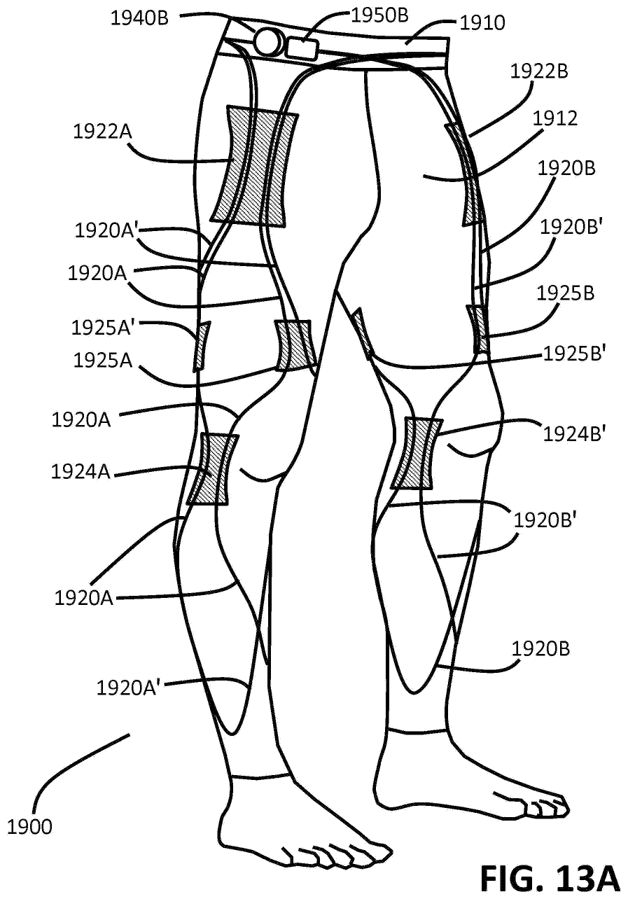

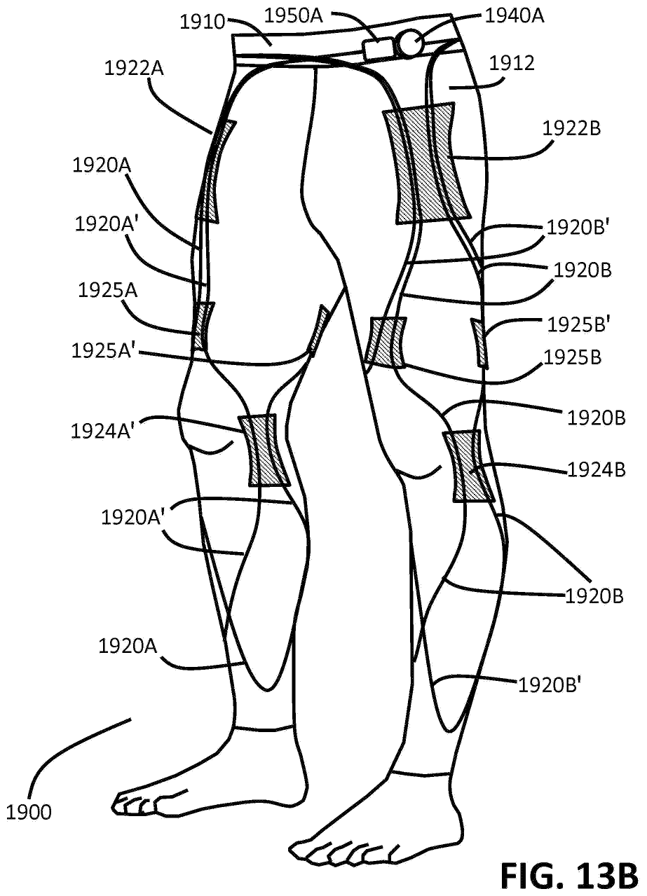

FIG. 13A and FIG. 13B show two different views of an embodiment of knitted brace garment 1900 arranged for enveloping at least in part a first joint (for example the right knee of the right leg) of a human user. Garment 1900 comprises knitted elastically extensible matrix fabric 1912 and a plurality of low elasticity web portions having a greater Young's modulus than the knitted elastically extensible matrix fabric 1912.

The term “knitted garment” is used herein to describe a garment created by the process of knitting in one or more stitch patterns, rather than, for example without limitation, weaving or extruding. It is to be understood that the garment of FIG. 13A and FIG. 13B may be knitted as two separate sections, one for each leg, which may then be joined together by a mechanism other than knitting. The term “panel” shall be used herein to describe any continuously knitted section comprising elements of differing properties, all knitted in one continuous process. In the example of FIG. 13A and FIG. 13B, a panel may be knitted for each leg and then joined to itself along a scam by a mechanism other than knitting to create a tubular leg portion. The term “knitted garment” is used herein to describe a garment of which the major panels, for example a panel for each leg, are knitted and the major panels are then joined together along seams by other mechanisms. A given major panel of the garment, complete with its low elasticity web portions and conduits described below, is created in one knitting process. This, in particular, differentiates the knitted garment from garments created from sheets, bolts or rolls of pre-made fabrics. Knitted brace garment 1900 cannot be created from sheets, bolts or rolls of pre-made fabrics.

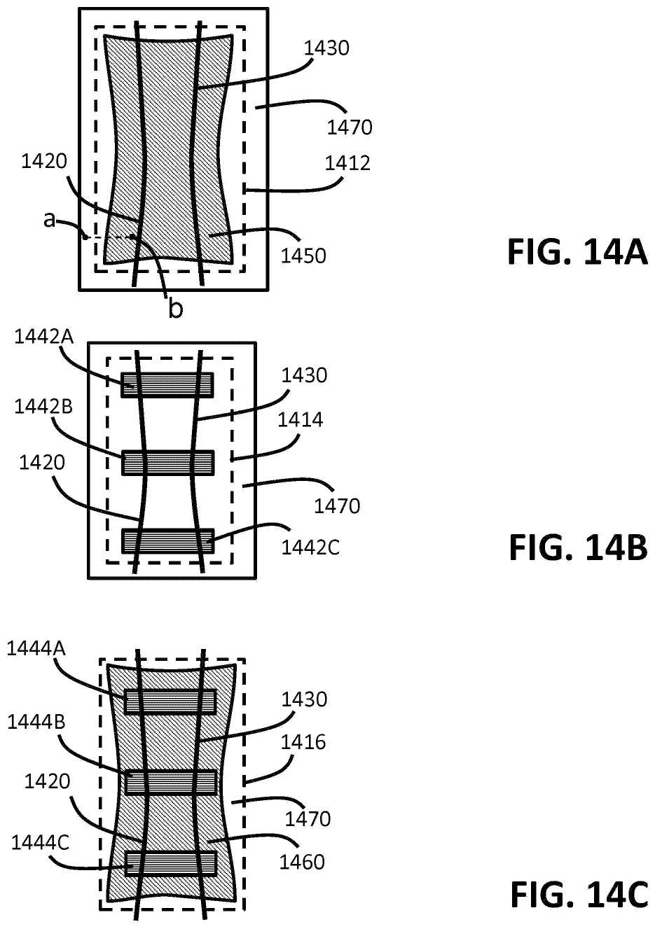

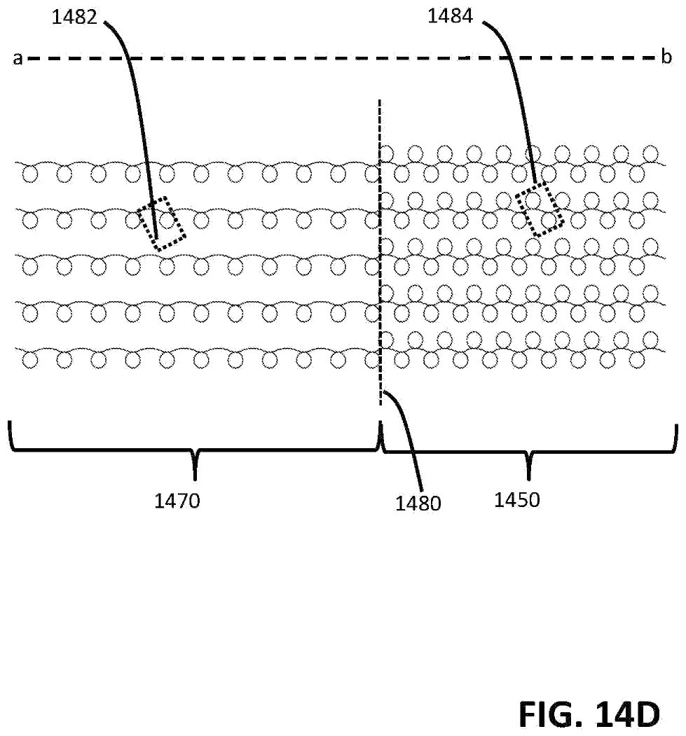

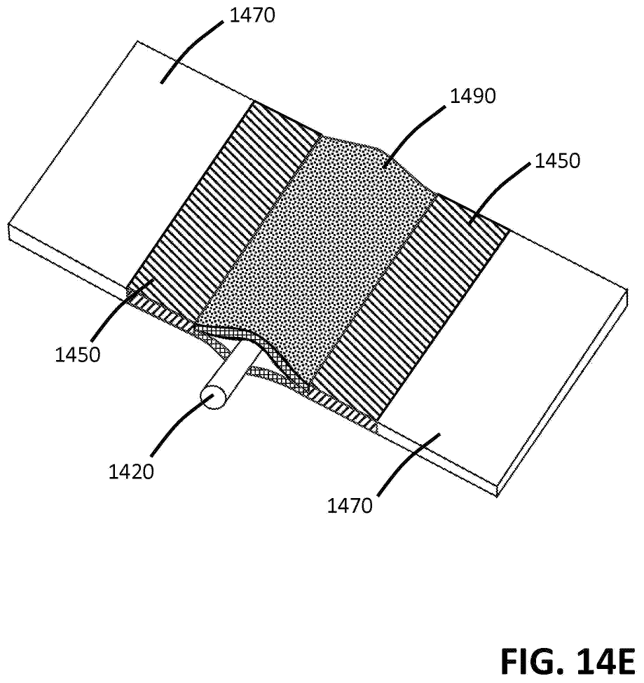

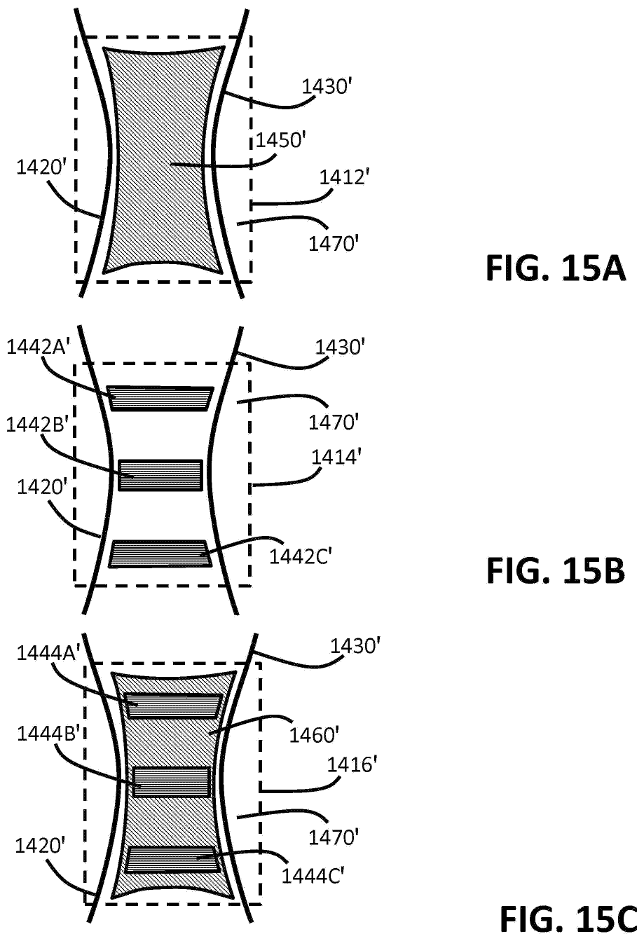

Ten low elasticity web portions 1922A, 1922B, 1924A, 1924B, 1924A′, 1924B′, 1925A, 1925B, 1925A′ and 1925B′ of FIG. 13A and FIG. 13B are all disposed and configured to guide substantially inextensible tension members as described in more detailed below. The term “guide portions” is used herein to describe ten low elasticity web portions 1922A, 1922B, 1924A, 1924B, 1924A′, 1924B′, 1925A, 1925B, 1925A′ and 1925B′. In more general embodiments, a guide portion of the garment may comprise more than one low elasticity web portion, as discussed below with reference to FIG. 14B, FIG. 14C, FIG. 15B and FIG. 15C.

Garment 1900 comprises two anterior thigh guide portions 1925A and 1925B disposed on the right and left thighs respectively, proximate and superior the anteriors of the corresponding knee regions of garment 1900 when worn by a user. Garment 1900 comprises two posterior thigh guide portions 1925A′ and 1925B′ disposed on the right and left thighs respectively, proximate and superior the posteriors of the corresponding kncc regions of garment 1900 when worn by a user. It also comprises two upper lateral guide portions 1922A and 1922B on the lateral sides of the wearer torso proximate the hip joints when garment 1900 is worn by a user. Garment 1900 also comprises two lateral knee guide portions 1924A and 1924B disposed proximate the lateral sides of respectively the right and left knee joints of the garment 1900 when worn by a user. Garment 1900 also comprises two medial knee guide portions 1924A′ and 1924B′ disposed proximate the medial sides of respectively the right and left knee joints of garment 1900 when worn by a user. Other embodiments may have different distributions of low elasticity guide portions.

In one embodiment, the various low elasticity web portions, being variously guide portions 1922A, 1922B, 1924A, 1924B, 1924A′, 1924B′, 1925A, 1925B, 1925A′ and 1925B′, may be knitted from the same unbroken strand of yarn as matrix fabric 1912 of garment 1900, but employing a different stitch pattern. We shall herein employ the term “zonal fabric” to describe the fabric of the low elasticity web portions defining, among other elements of garment 1900, the various guide portions. The difference between matrix fabric 1912 and the zonal fabric of the low elasticity web portions may be that the former has a first stitch pattern and the latter has a second stitch pattern. The second stitch pattern may be selected to result in a higher Young's modulus for the zonal fabric. In one embodiment, the zonal fabric of the various low elasticity web portions may be knitted from the same yarn as matrix fabric 1912 of garment 1900, but employing a different strand of yarn. In one embodiment, the zonal fabric of the various low elasticity web portions may be knitted from two or more strands of the same yarn as matrix fabric 1912 of garment 1900. In one embodiment, the zonal fabric of the various low elasticity web portions may be knitted from a different type of yarn than matrix fabric 1912 of garment 1900. In one embodiment, the zonal fabric of the various low elasticity web portions may be knitted from a yarn of a different type of material than matrix fabric 1912 of the garment 1900. In some embodiments, the zonal fabric of the various low elasticity web portions may be knitted using combinations of these methods.

Six of the ten guide portions described with reference to FIG. 13A and FIG. 13B, being guide portions 1922A, 1922B, 1924A, 1924B, 1924A′, 1924B′, have an additional function of bracing articulating joints of the wearer when garment 1900 is worn by the wearer. These six guide members will be more particularly referred to herein as “braces”. These six braces will now be described in more detail, starting with the four braces directed toward stabilizing the knee joints, followed by a description of the two braces directed toward stabilizing the hip joints.

To protect the knee joint of a wearer, garment 1900 of FIG. 13A and FIG. 13B comprises two lateral knee guide portions 1924A and 1924B disposed on the lateral sides of respectively the right and left knee joints of garment 1900 when worn by a user. Medially directed pressure is applied to the right and left knee joints via respectively lateral knee guide portions 1924A and 1924B under the action of tension members 1920A and 1920B respectively. This stabilizes the two knee joints against lateral displacement as a result of problems with the lateral collateral ligaments of the two knees. Low elasticity web portions 1924A and 1924B serve as lateral knee braces. The configurations of tension members are described in more detail below.

To further protect the knee joint of a wearer, garment 1900 of FIG. 13A and FIG. 13B comprises two medial knee guide portions 1924A′ and 1924B′ disposed on the medial sides of respectively the right and left knee joints of garment 1900 when worn by a user. Laterally directed pressure is applied to the right and left knee joints via respectively medial knee guide portions 1924A′ and 1924B′ under the action of tension members 1920A′ and 1920B′ respectively. This stabilizes the two knee joints against medial displacement as a result of problems with the medial collateral ligaments of the two knees. Low elasticity web portions 1924A′ and 1924B′ serve as medial knee braces. The configurations of tension members are described in more detail below.

In the embodiment of FIG. 13A and FIG. 13B, two tension members 1920A and 1920A′ extend in three-dimensional paths within knitted conduits about the right leg and torso of garment 1900. Tension members 1920B and 1920B′ correspondingly extend in three-dimensional paths within knitted conduits about the left leg and torso of garment 1900. In the embodiment of FIG. 13A and FIG. 13B, each of the tension members is a closed loop about the corresponding leg. In the embodiment of FIG. 13A and FIG. 13B, two segments of tension member 1920A are shown traversing low elasticity web portion 1924A along curved paths such that the two segments are further apart from each other at the opposing upper and lower edges of web portion 1924A than nearer the middle of web portion 1924A. When longitudinal tension is applied to tension member 1920A, tension member 1920A is tightened around the right leg and torso and a medially directed force is developed against the lateral side of the right knee by the low elasticity web portion 1924A. Correspondingly, when longitudinal tension is applied to tension member 1920B, tension member 1920B is tightened around the left leg and torso and a medially directed force is developed against the lateral side of the left knee by the low elasticity web portion 1924B.

In the embodiment of FIG. 13A and FIG. 13B, two segments of tension member 1920A′ are shown traversing low elasticity web portions 1924A′ along curved paths such that the two segments are further apart from each other at the opposing upper and lower edges of web portion 1924A′ than nearer the middle of web portion 1924A′. When longitudinal tension is applied to tension member 1920A′, tension member 1920A′ is tightened around the right leg and torso and a laterally directed force is developed against the medial side of the right knee by low elasticity web portion 1924A′. Correspondingly, when longitudinal tension is applied to tension member 1920B′, tension member 1920B′ is tightened around the left leg and torso and a laterally directed force is developed against the medial side of the left knee by low elasticity web portion 1924B′.

To protect the hip joints of a wearer, garment 1900 of FIG. 13A and FIG. 13B comprises two upper lateral guide portions 1922A and 1922B disposed on the lateral sides of respectively the right and left hip joints of garment 1900 when worn by a user. Medially directed pressure is applied to the right and left hip joints via respectively upper lateral hip guide portions 1922A and 1922B under the action of tension members 1920A and 1920B respectively as well as tension members 1920A′ and 1920B′. This stabilizes the two hip joints, restricting the femur from moving toward the midline of the body. Low elasticity web portions 1922A and 1922B, whilst serving as guide portions for tension members 1920A, 1920A′, 1920B and 1920B′, also serve as hip braces for respectively the right and left hip. As already described with respect to knee braces 1924A, 1924B, 1924A′, 1924B′, braces 1922A and 1922B, also function on the basis of tension members 1920A, 1920A′, 1920B and 1920B′ following curved paths as they traverse the low elasticity web portions that form the basis of braces 1922A and 1922B. In the case of hip braces 1922A and 1922B, the tension in the braces and the consequent pressure on the hips is based on two pairs of tension member segments traversing braces 1922A and 1922B.

In general, other three-dimensional paths are contemplated for the tension members and in some of those embodiments the tension members are not required to be closed loops about any part of the anatomy. In general, the two segments of tension member(s) traversing a low elasticity web portion is a requirement for the low elasticity web portion to function as a guide portion, but it is not a requirement that the two segments of tension member should be segments of the same tension member. It is only a requirement that there be longitudinal tension in the two segments of tension member so as to induce tension in the low elasticity web portion.

FIG. 13A and FIG. 13B show in total four tension members 1920A, 1920A′, 1920B and 1920B′. In other embodiments there may be a need for only a single low elasticity web portion, for example low elasticity web portion 1924A. As may be seen in FIG. 13A and FIG. 13B, pressure is applied to low elasticity web portion 1924A only by tension member 1920A. Consequently, embodiments are contemplated in which garment 1900 comprises a single medial or lateral knee low elasticity web portion and a single corresponding tension member.

Tension member 1920A and guide portion 1924A are described below, understanding therewith that the same principles and considerations hold for low elasticity web portions 1924A′, 1924B, 1924B′, 1922A, 1922B, 1925A, 1925A′, 1925B, 1925B′ and tension members 1920A′, 1920B and 1920B′. In embodiments in which tension member 1920A is disposed on elastically extensible matrix fabric 1912 of garment 1900, tension member 1920A may be disposed within conduits of the types already described with reference to FIGS. 10A, 10B and 10C. However, in those embodiments in which tension member 1920A is disposed within elastically extensible matrix fabric 1912 or within low elasticity web portions 1924A′, 1924B, 1924B′, 1922A, 1922B, 1925A, 1925A′, 1925B, 1925B′, it may be disposed in conduits knitted into elastically extensible matrix fabric 1912 or into low elasticity web portions 1924A′, 1924B, 1924B′, 1922A, 1922B, 1925A, 1925A′, 1925B, 1925B′ of garment 1900 respectively.

In one embodiment, the conduits are knitted from the same strand of yarn as elastically extensible matrix fabric 1912, but using a different stitch pattern than that employed for elastically extensible matrix fabric 1912. This different third stitch pattern may be used to delineate the conduits for tension member 1920A within the matrix that is otherwise that of elastically extensible matrix fabric 1912. Hereby, the difference between the conduits is not the yarn employed, but the fact that matrix fabric 1912 has a first stitch pattern and the conduits have a third stitch pattern. In some embodiments, the conduits may be knitted from the same yarn as matrix fabric 1912 of garment 1900, but employing a different strand of yarn. In one embodiment, the conduits may be knitted from a different type of yarn than matrix fabric 1912 of garment 1900. In one embodiment, conduits may be knitted from a yarn of a different type of material than matrix fabric 1912 of garment 1900. In some embodiments, conduits may cross boundaries between matrix fabric and the zonal fabric of the low elasticity web portions. A fourth stitch pattern may be used to further delineate the conduits for the tension member 1920A within the low elasticity web portion.

Tensioner 1940A may be disposed on belt 1910 of garment 1900 in the three-dimensional path of tension member 1920A about the right leg and torso of garment 1900 and may be arranged to adjust a tension in tension member 1920A. A corresponding tensioner 1940B, may be disposed on belt 1910 of garment 1900 in the three-dimensional path of tension member 1920B about the left leg and torso of garment 1900 and may be arranged to adjust a tension in tension member 1920B. Similar tensioners may also be disposed on the belt to adjust tension in the other tension members 1920A′, 1920B and 1920B′. Suitable tensioners that may be employed include, without limitation, dials, cord locks, drawstrings, laces and straps and may be placed anywhere along the path of the corresponding tension member.

Tension member 1920A may comprise a plurality of different strands. Tension evener 1950A may be disposed on belt 1910 of garment 1900 in the three-dimensional path of tension member 1920A about the right leg and torso of garment 1900 and may be arranged to even a tension distribution among different strands in tension member 1920A. Tension evener 1950B may correspondingly be disposed on belt 1910 of garment 1900 in the three-dimensional path of tension member 1920B about the left leg and torso of garment 1900 and may be arranged to even a tension distribution among different strands in tension member 1920B. Suitable tension eveners and their use have already been described in U.S. patent application Ser. No. 17/222,903 of Apr. 5, 2021, which is incorporated herein in full. In embodiments with more than one tension member per limb, corresponding additional tension eveners may be provided. Tensioners 1940A and 1940B, as well as tension eveners 1950A and 1950B may be disposed in line with the tension members on portions of garment 1900 other than belt 1910.

The behaviors and effects of low elasticity web portions 1924A, 1924A′, 1924B, 1924B′, 1922A, 1922B, 1925A, 1925A′, 1925B, and 1925B′ are dictated by the relative Young's modulus of the Web portions and of matrix fabric 1912, the shapes of the low elasticity web portions, their placement, and the exact routes of any tension members traversing them or extending proximate them.

Any guide portion may comprise a plurality of low elasticity web portions, and each low elasticity web portion may or may not have a different elastic property, based on, for example without limitation, stitch type, yarn type and/or material of the yarn. FIGS. 14A, 14B and 14C show three different embodiments of generalized guide portion 1412, 1414 and 1416 respectively for use in brace garments of the type shown in FIG. 13A and FIG. 13B. In each of the three embodiments, the relevant guide portion is surrounded by matrix fabric 1470 of the garment and is traversed by tension members 1420 and 1430. Tension members 1420 and 1430 have curved paths through guide portions 1412, 1414 and 1416 such that tension members 1420 and 1430 are separated by a greater distance from each other at their exit points from guide portions 1412, 1414 and 1416 than at the centers of guide portions 1412, 1414 and 1416. When longitudinal tension is applied to tension members 1420 and 1430, tension develops within guide portions 1412, 1414 and 1416 between tension members 1420 and 1430. It should be noted that guide portions 1412, 1414 and 1416 are not fashioned on top of matrix fabric 1470, but are knitted into it with the knitting being continuous across at least some boundaries between matrix fabric 1470 and any of the low elasticity web portions of which guide portions 1412, 1414 and 1416 may be comprised.

FIG. 14A shows low elasticity web portion 1450 comprising one contiguous section of just one single first zonal fabric knitted into the matrix fabric to form guide portion 1412. FIG. 14B shows guide portion 1414 comprising three separate low elasticity web portions 1442A, 1442B and 1442C of a second zonal fabric knitted into matrix fabric 1470 of a garment. In general, low elasticity web portions 1442A, 1442B and 1442C may each consist of a different zonal fabric. FIG. 14C shows guide portion 1416 comprising one discontiguous low elasticity web portion 1460 of first zonal fabric in which is embedded by knitting three low elasticity web portion 1444A, 1444B and 1444C of a second zonal fabric, the entire compound low elasticity web portion being knitted into matrix fabric 1470. Each of these three implementations of web portion 1924A will behave differently when tension is applied or changed within tension members 1420 and 1430. In general, low elasticity web portions 1444A, 1444B and 1444C may each consist of a different zonal fabric.

FIG. 14D shows an example change in stitch pattern, continuously knitted along line a-b in FIG. 14A. Seamless boundary 1480 between matrix fabric 1470 and the fabric of low elasticity web portion 1450 is shown in FIG. 14D. In the case of matrix fabric 1470, a repeatable unit cell of the stitch pattern is given by the loop structure in block 1482. In the case of matrix fabric 1470, a stitch pattern of the fabric of low elasticity web portion 1450 is given by the loop structure in block 1484. Matrix fabric 1470 and low elasticity web portion 1450 are continuously knitted using the same yarn, but using differing stitches. The stitch in block 1484 employed in low elasticity web portion 1450 results in a fabric that is denser than matrix fabric 1470 and imparts to low elasticity web portion 1450 a higher Young's Modulus and thereby a lowered elasticity as compared with matrix fabric 1470. In other embodiments, multiple strands of yarn may be employed and in yet further embodiments the yarns may differ and/or be of different materials. The stitch patterns may also differ, as in the case of FIG. 14D, but boundary 1480 between matrix fabric 1470 and low elasticity web portion 1450 is seamless in all embodiments.

On the basis of the non-limiting examples of FIGS. 14A, 14B and 14C, it may be seen that a generalized guide portion, such as for example guide portions 1922A, 1922B, 1924A, 1924B, 1924A′, 1924B′, 1925A, 1925B, 1925A′ and 1925B′ of FIG. 13A and FIG. 13B, may comprise at least one low elasticity web portion knitted into matrix fabric 1912 of garment 1900. In general, a guide portion may comprise a plurality of low elasticity web portions, including one or more low elasticity web portions surrounded by another low elasticity web portion of a different zonal fabric, all knitted into the garment such that the knitting across any boundary between low elasticity web portions, or between any low elasticity web portion and the matrix fabric of the garment, is continuous. Wherever there is a junction between a zonal fabric and the matrix fabric, that junction is formed by continuous knitting across the perimeter of the zonal fabric. The stitch patterns and even the yarns of the zonal fabric and matrix fabrics may differ, but the knitting process is continuous across the boundary between the two fabrics. The term “continuously knitted” is used herein to describe the process for so creating a boundary between zonal fabric and matrix fabric in garment 1900, or between two portions of different zonal fabrics and the boundary so created is referred to herein as a “continuously knitted boundary”.

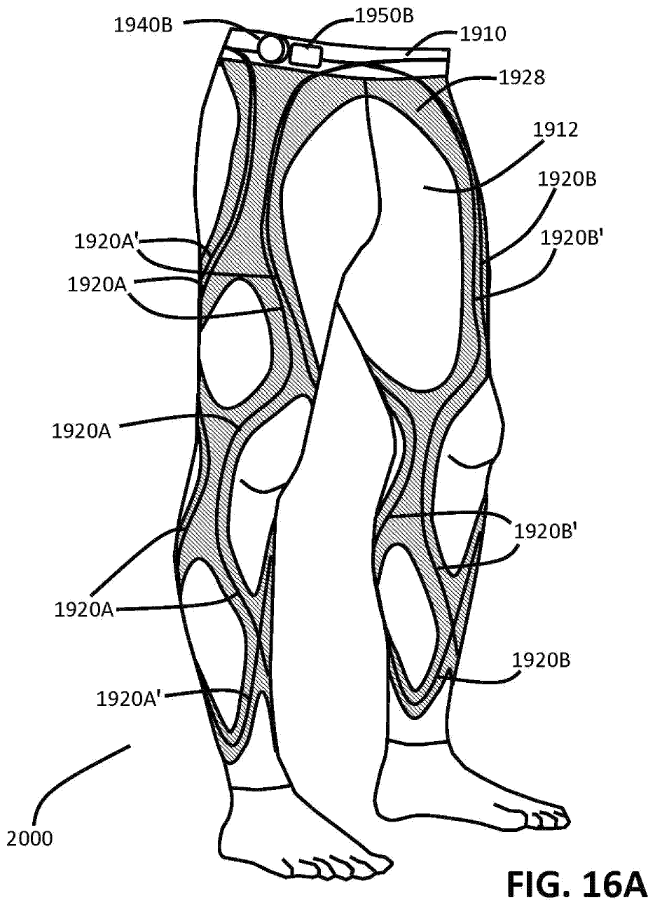

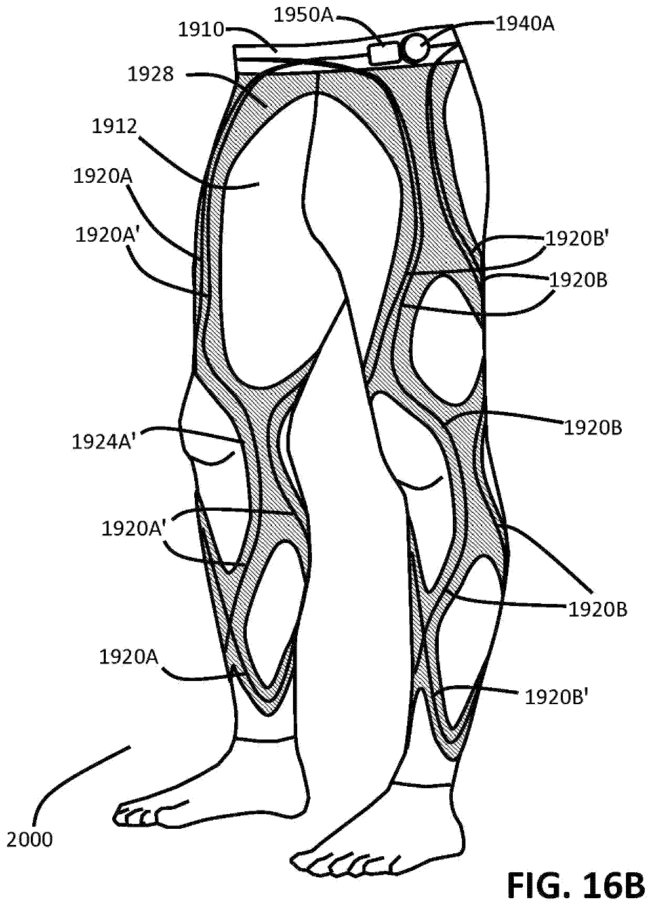

FIG. 14E shows a representative portion of a brace garment of the types described with reference to FIGS. 13A and 13B, as well as FIGS. 16A to 24B, described below. Each of the embodiments in the listed figures has a knitted matrix fabric 1470 and at least one low elasticity web portion 1450. The representative portion shows an inextensible tension member 1420 extending along a conduit 1490 knitted through the low elasticity web portion 1450 in FIG. 14E. In other areas of FIGS. 13A, 13B, and FIGS. 16A to 24B, the conduit 1490 may be knitted to extend through matrix fabric 1470 instead of through a low elasticity web portion 1450. In FIGS. 13A, 13B, and FIGS. 16A to 24B, the conduits, being rather narrow on the scale of the entire garments, are not shown. At least some of the boundaries between the low elasticity web portions 1450 and the matrix fabric 1470 may be continuously knitted boundaries, and the same is true of boundaries between knitted conduits 1490 and either or both of the matrix fabric 1470 and the low elasticity web portions 1450.

FIGS. 13A, 13B, 14A, 14B and 14C show all low elasticity web portions traversed by either one or two tension members. In general, the tension members are not restricted to traversing the low elasticity web portions of garment 1900 and, in a more general embodiment, the tension members need only extend proximate the low elasticity web portions. FIGS. 15A, 15B and 15C show further embodiments for the mutual disposition of low elasticity web portions and tension members. In these embodiments, tension members 1420′ and 1430′ do not physically traverse low elasticity web portions 1450′, 1442A′, 1442B′, 1442C′, 1444A′, 1444B′, 1444C′ and 1460′, but, instead, tension members 1420′ and 1430′ are disposed proximate edges of low elasticity web portions 1450′, 1442A′, 1442B′, 1442C′ and 1460′. The arrangements shown in FIGS. 15A, 15B and 15C nevertheless still cause the forces on the tension members to be conveyed to the low elasticity web portions, albeit via small portions of matrix fabric 1970′. Tension members 1420′ and 1430′ may be viewed as tethered to low elasticity web portions 1450′, 1442A′, 1442B′, 1442C′ and 1460′ by means of garment fabric 1970′. In such embodiments, the regions of elastically extensible matrix fabric of the garment disposed between the tension member conduits and the low elasticity web portions stretch under the action of the opposing forces pulling the tension members apart. The tension in all fabric between the two tension members is the same, but the strain is largely taken up in the lower Young's modulus matrix fabric 1970′.

FIG. 16A and FIG. 16B show an embodiment of knitted brace garment 2000, in which the plurality of low elasticity web portions of FIG. 13A and FIG. 13B have been substituted with a single low elasticity web portion 1928 knitted as a single contiguous portion of one zonal fabric. In FIG. 16A and FIG. 16B, we retain all the same numberings for the other elements of brace garment 1900 of FIG. 13A and FIG. 13B. In the embodiment of FIG. 16A and FIG. 16B, low elasticity web portion 1928 is arranged to house tension members 1920, 1920A′, 1920B and 1920B′ at least in part along their three-dimensional paths so that tension members 1920, 1920A′, 1920B and 1920B′ lic within low elasticity web portion 1928. In the embodiments of FIG. 13A, FIG. 13B, FIG. 16A and FIG. 16B, belt 1910 may in general be knitted as part of the matrix fabric of garment 1900 and 2000. In other embodiments, belt 1910 may be knitted to have a zonal fabric.

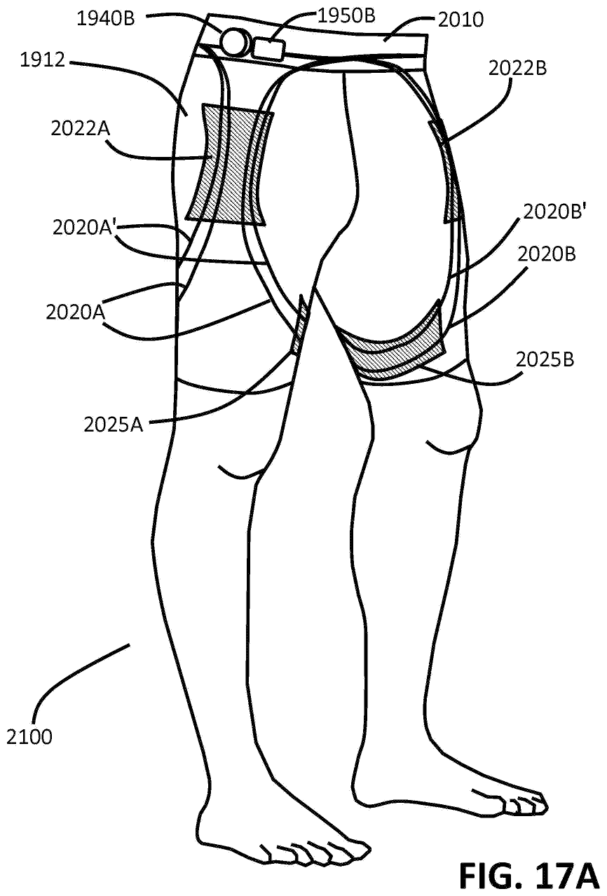

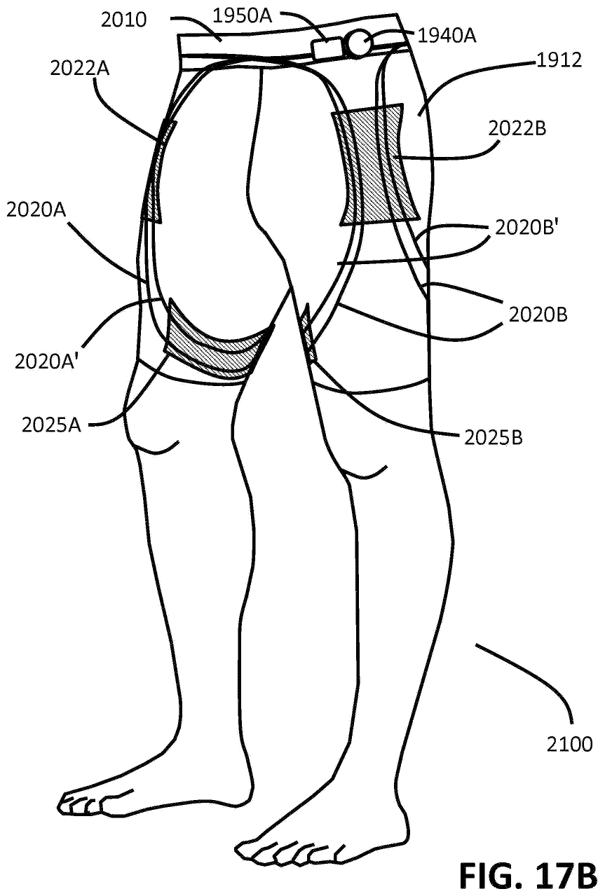

FIG. 17A and FIG. 17B show brace garment 2100 for the human hip, being a version of the brace garment of FIG. 13A and FIG. 13B in which the two legs of the brace garment have been shortened to above the knee. Brace garment 2100 has only four low elasticity guide portions, namely 2022A and 2022B serving as braces proximate the right and left hip joints respectively, low elasticity guide portion 2025A guiding tension members 2020A and 2020A′ around the inner right thigh and low elasticity guide portion 2025B guiding tension members 2020B and 2020B′ around the inner left thigh. As in the case of garment 1900 of FIG. 13A and FIG. 13B, the tension in tension members 2020A, 2020A′, 2020B and 2020B′ induces tension in braces 2022A and 2022B, which leads to medially directed pressure on the two hip joints. Garment 2100 may also have tensioners 1940A and 1940B, as well as tension eveners 1950A and 1950B with the functions already described. Belt 2010 may in general be knitted as part of the matrix fabric of garment 2100. In other embodiments, belt 2010 may be knitted to have a zonal fabric. FIG. 17A and FIG. 17B show brace garment 2100 as having two tension members per leg. In other embodiments there may be either a single tension member per leg, or a greater plurality of tension members.

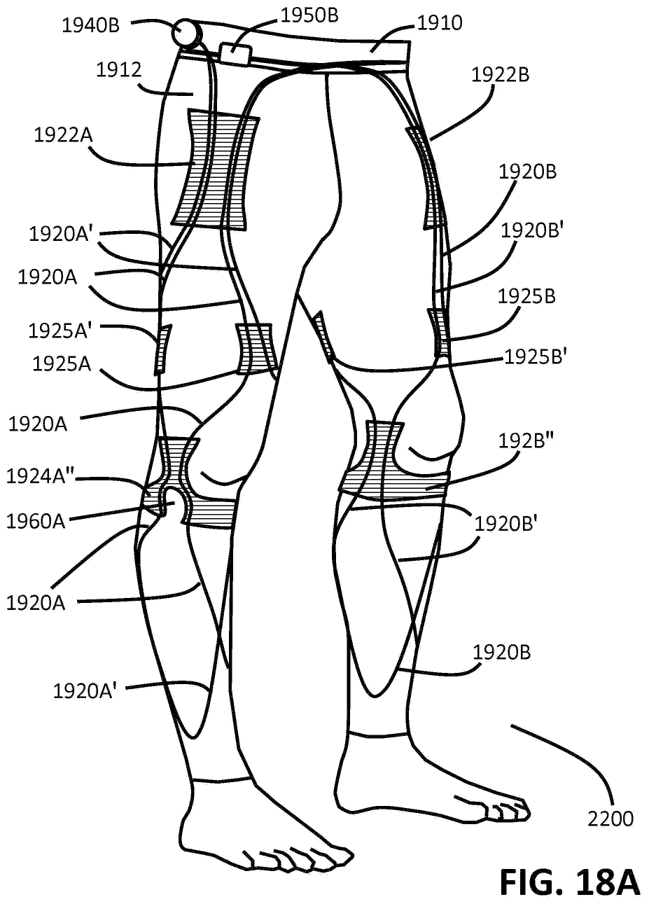

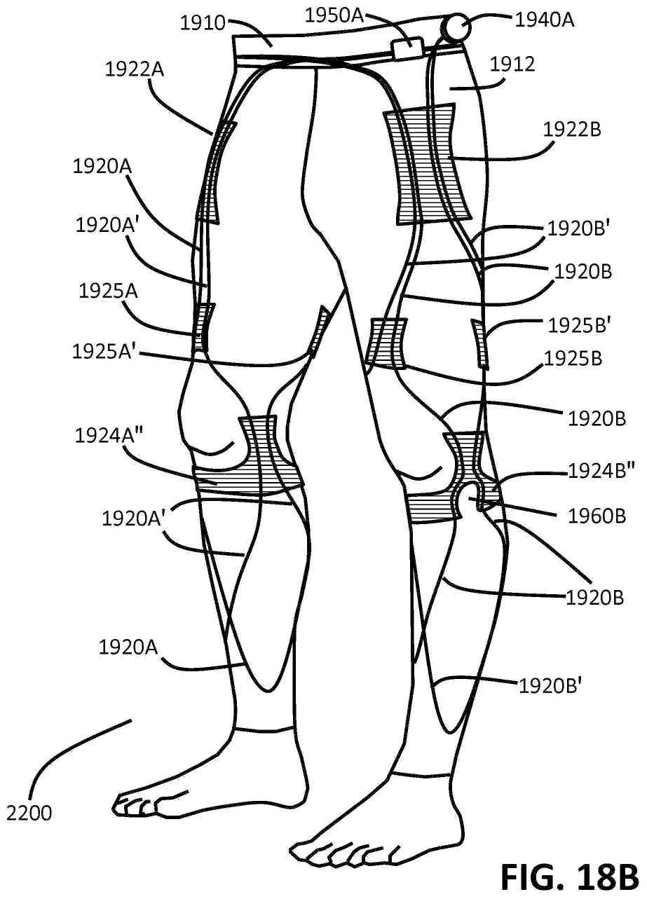

FIG. 18A and FIG. 18B show a variation 2200 on garment 1900 of FIG. 13A and FIG. 13B, the only difference being low elasticity web portions 1924A″ and 1924B″ in which tension members 1920A′ and 1920B′ respectively are routed by conduits to avoid areas 1960A and 1960B respectively where the right and left peroneal nerves of the user pass proximate the head of the right and left fibulas of the user respectively. Pressure on the sensitive peroneal nerve may lead to discomfort, pain or even permanent damage to that nerve. In garment 2200, as shown in FIG. 18A and FIG. 18B, low elasticity web portions 1924A″ and 1924B″ may be shaped to not cover the heads of the fibulas of the user. Similarly, the tension members may be routed to avoid other superficial peripheral nerves where pressure may lead to discomfort, pain or even permanent damage to that nerve. This principle may be applied in general to other sensitive areas of the user's anatomy where pressure is ideally to be avoided. In some embodiments the cables may be routed to avoid bony prominences such as the medial and lateral malleolus where pressure from the tension members may be uncomfortable. In other embodiments, the cables may be routed to avoid a sensitive surgical incision site such as an Anterior Cruciate Ligament reconstruction incision site where pressure from the tension members may be uncomfortable.

FIGS. 13A to 18B describe knitted brace garment 1900, 2000, 2100, 2200 configured for enveloping at least a first joint of a human user when the garment is worn by the user, the garment comprising: elastically extensible matrix fabric 1912; at least one low elasticity web portion 1924A, 1924A′, 1924A″, 1924B, 1924B′, 1924B″, 1922A, 1922B, 1925A, 1925A′, 1925B, 1925B′, 1928 having a first zonal fabric different from the matrix fabric; and at least two segments of at least one tension member 1920A, 1920A′, 1920B, 1920B′, 2020A, 2020A′, 2020B, 2020B′ disposed to induce tension in the at least one low elasticity web portion, wherein at least one boundary between the at least one low elasticity web portion and matrix fabric 1912 is a continuously knitted boundary.

The at least one tension member 1920A, 1920A′, 1920B, 1920B′, 2020A, 2020A′, 2020B, 2020B′ may be disposed along a path that spatially relates the at least one tension member to at least one natural ligament of the knee of the user, wherein at least one low elasticity web portion 1924A, 1924A′, 1924A″, 1924B, 1924B′, 1924B″, 1922A, 1922B, 1925A, 1925A′, 1925B, 1925B′, 1928 is disposed to apply pressure to one of a lateral side and a medial side of the knee of a wearer for bracing the knee. As described with reference to FIG. 18A and FIG. 18B, the brace garment 2100, in one embodiment, may be configured for enveloping a hip joint of a corresponding leg of a human user when garment 2100 is worn by the user.

The brace garment may comprise continuously knitted conduits for guiding the at least one tension member wherein the conduits are knitted into at least one of the matrix fabric and the at least one low elasticity web portion. At least one of the conduits may extend along a curved path arranged to circumnavigate in part a sensitive feature of a body of a user when the garment is worn by the user, for example the peroneal nerve of a leg of the user.

The brace garment may comprise conduits disposed on top of matrix fabric 1912 and low elasticity web portions 1924A, 1924A′, 1924A″, 1924B, 1924B′, 1924B″, 1922A, 1922B, 1925A, 1925A′, 1925B, 1925B′, 1928 to guide at least one tension member 1920A, 1920A′, 1920B, 1920B′, 2020A, 2020A′, 2020B, 2020B′. The at least one tension member may be a closed loop. The at least one tension member may be anchored to the triceps surae of the user by a loop at least in part about the triceps surae. The brace garment may comprise tensioner 1940A, 1940B on garment 1900, 2000, 2100, 2200 arranged for adjusting a tension in the at least one tension member. The at least one tension member may comprise a plurality of strands; and the garment may comprise tension evener 1950A, 1950B arranged for evening tension among the plurality of strands. The garment may comprise belt 1910, 1920 and the at least one tension member may have a proximal end and a distal end, the proximal end of the at least one tension member being anchored to belt 1910, 1920 and the distal end of the at least one tension member being disposed to be anchored by a triceps surae.

The at least two segments of at least one tension member 1920A, 1920A′, 1920B, 1920B′, 2020A, 2020A′, 2020B, 2020B′ may be disposed to longitudinally extend along curved paths that traverse the at least one low elasticity web portion 1924A, 1924A′, 1924A″, 1924B, 1924B′, 1924B″, 1922A, 1922B, 1925A, 1925A′, 1925B, 1925B′, 1928. The curved paths may curve in opposing directions to induce tension in the at least one low elasticity web portion. The at least two segments of the at least one tension member may be disposed to longitudinally extend along a curved paths through matrix fabric 1912 proximate two opposing boundaries between the at least one low elasticity web portion and the elastically extensible matrix. The curved paths may curve in opposing directions to induce tension in the at least one low elasticity web portion. Two segments of the at least one tension member may extend along two corresponding curved paths traversing the at least one low elasticity web portion to induce tension between the two segments of the at least one tension member.

Each of the at least one low elasticity web portions 1924A, 1924A′, 1924A″, 1924B, 1924B′, 1924B″, 1922A, 1922B, 1925A, 1925A′, 1925B, 1925B′, 1928 may comprise at least one portion of the first zonal fabric knitted into matrix fabric 1912. As shown in FIGS. 14C and 15C, at least one low elasticity web portion (1416 and 1416′ respectively) may comprise at least one section (1444B and 1444B′ respectively) of a second zonal fabric having a continuously knitted boundary with at least one section of the first zonal fabric. As per FIGS. 16A and 16B, brace garment 2000 may comprise at least one low elasticity web portion 1928 disposed to partially envelop at least one limb of the user.

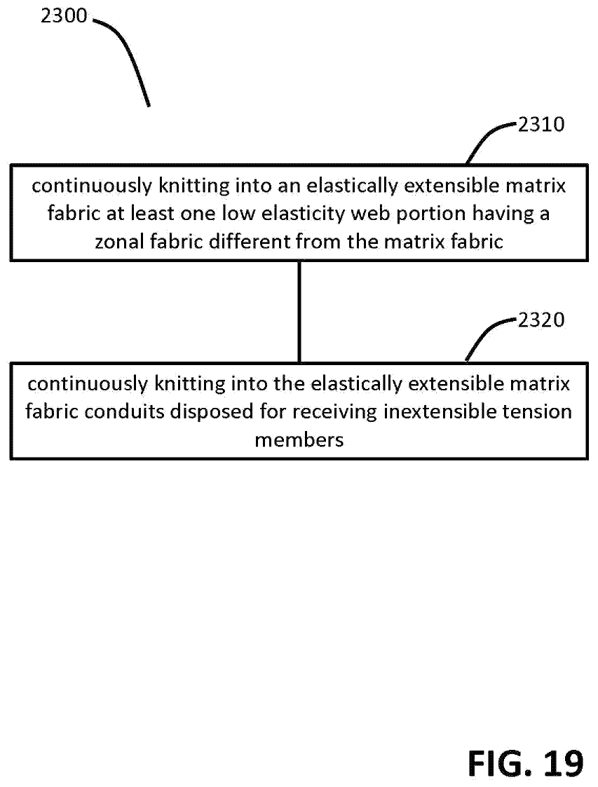

In a further aspect described with reference to FIG. 19, method is provided for creating brace garment 1900, 2000, 2100, 2200 for enveloping at least a first joint of a human user when the garment is worn by the user, the method comprising continuously knitting into elastically extensible matrix fabric 1912 at least one low elasticity web portion 1924A, 1924A′, 1924A″, 1924B, 1924B′, 1924B″, 1922A, 1922B, 1925A, 1925A′, 1925B, 1925B′, 1928 having a zonal fabric different from matrix fabric 1912; and continuously knitting into the elastically extensible matrix fabric conduits disposed for receiving inextensible tension members 1920A, 1920A′, 1920B, 1920B′, 2020A, 2020A′, 2020B, 2020B′, wherein the tension members when received in the conduits are disposed to induce tension in the at least one low elasticity web portion, and wherein at least one boundary between the at least one low elasticity web portion and matrix fabric 1912 is a continuously knitted boundary.

The zonal fabric may be continuously knitted from the same yarn as the matrix fabric. The zonal fabric may be continuously knitted from a plurality of strands of the same yarn as the matrix fabric. The matrix fabric may be knitted using a first stitch pattern and the zonal fabric is continuously knitted from the same yarn as the matrix fabric using a second stitch pattern different from the first stitch pattern. The zonal fabric may be continuously knitted from one or more different yarns from the yarn of the matrix fabric. The zonal fabric may be continuously knitted from the same yarn as the as the matrix fabric and from one or more yarns that differ from the yarn of the matrix fabric. The matrix fabric may be knitted using a first stitch pattern and the zonal fabric is continuously knitted using a second stitch pattern different from the first stitch pattern.

The conduits may be continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

The zonal fabric may be continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

In one embodiment, the different yarn may be a melting yarn. Melting yarns are designed to melt when heat is applied after the knitting process resulting in a higher Young's modulus of the zonal fabric.

FIGS. 2A to 18B employ the example of the human leg and lower body to describe knitted brace garments for stabilizing a human knee and hip. The concepts described herein are, however, not limited to the human knee and hip. The concepts may be applied to any anatomical joint to be stabilized, including for example without limitation, a human ankle, elbow, wrist, shoulder, neck, spine, and one or more vertebrae.

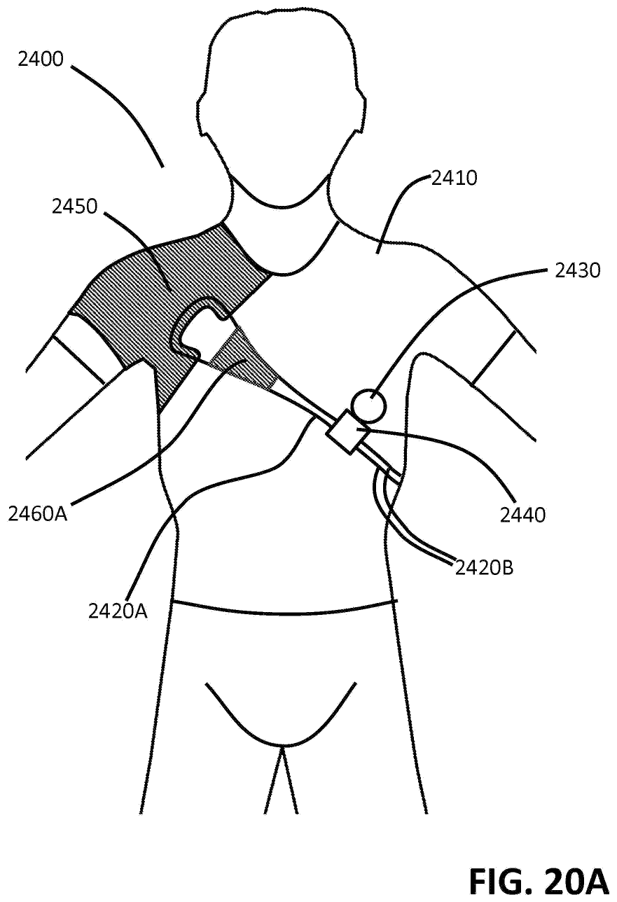

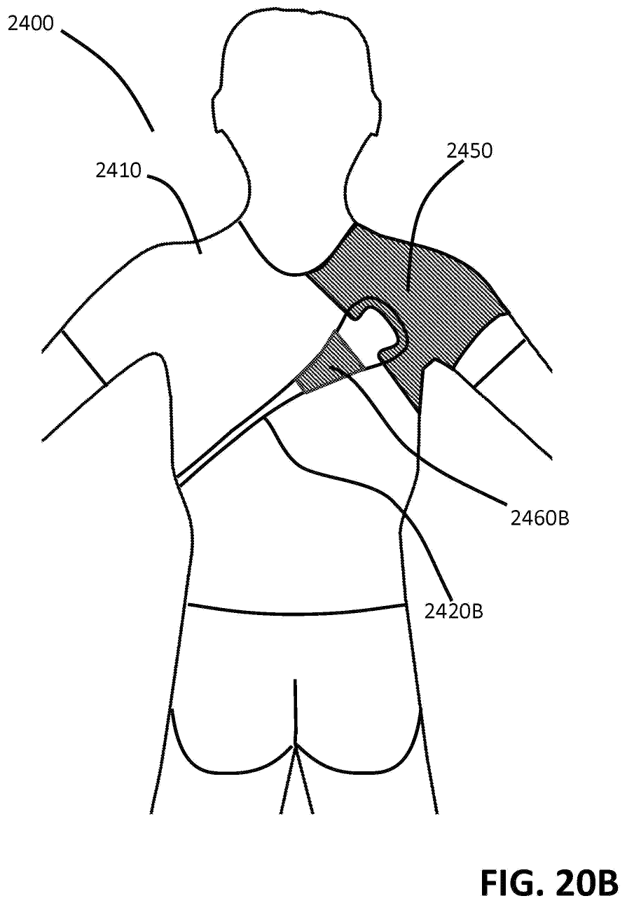

FIGS. 20A and 20B show a shoulder brace garment 2400 comprising a shirt 2410 of a knitted matrix fabric having a knitted low elasticity web portion 2450 disposed to brace the shoulder joint when placed under tension. Tension is applied to the low elasticity web portion 2450 by means of two inextensible tension members 2420A and 2420B engaged with low elasticity web portion 2450 and tensioned by tensioner 2430 via a tension evener 2440. Tension evener 2440 may be, for example without limitation, any of the tension eveners described in published PCT application WO2021203191A1. Tension members 2420A and 2420B may be guided by low elasticity guide portions 2460A and 2460B. Inextensible tension members 2420A and 2420B may be cables or strands arranged to move in knitted conduits within the low elasticity web portions 2450, 2460A and 2460B, and in the knitted matrix fabric of the shirt 2410, as already described with reference to FIG. 14E. At least some of the boundaries between the low elasticity web portions 2450, 2460A and 2460B, and the matrix fabric of the shirt 2410 may be continuously knitted boundaries.

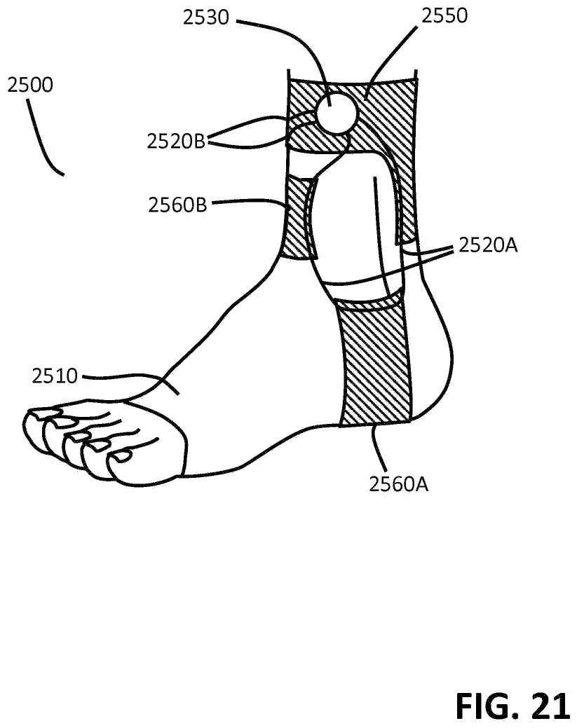

FIG. 21 shows a lateral side view of an ankle joint brace garment 2500 for a left foot comprising a hose 2510 of a knitted matrix fabric having a knitted low elasticity web portions 2560A and 2560B disposed to brace the ankle joint when placed under tension. For this example, the left leg is chosen. In other embodiments, the toeless hose 2510 may be a complete sock covering the toes. Tension is applied to the low elasticity web portions 2560A and 2560B by means of two inextensible tension members 2520A and 2520B engaged with the low elasticity web portions 2560A and 2560B and tensioned by tensioner 2530. Low elasticity web portion 2560A extends under the foot and is engaged by inextensible tension member 2520B on the medial side of the left foot in the same fashion as what inextensible tension member 2520A engages low elasticity web portion 2560A on the lateral side of the left foot. Tensioner 2530 may optionally apply tension to inextensible tension members 2520A and 2520B via a tension evener (not shown). The tension evener may be, for example without limitation, any of the tension eveners described in published PCT application WO2021203191A1. Tensioner 2530 may be disposed on an anchor band 2550 disposed about the lower leg above the ankle. Anchor band 2550 is employed as a stable base relative to which tension may be applied to low elasticity web portions 2560A and 2560B. Anchor band 2550 may also be knitted to be a low elasticity web portion. Tension members 2520A and 2520B may be guided by low elasticity web portions 2550, 2560A and 2560B. Inextensible tension members 2520A and 2520B may be cables or strands arranged to move in knitted conduits within low elasticity web portions 2550, 2560A and 2560B and in the knitted matrix fabric of the hose 2510, as already described with reference to FIG. 14E. At least some of the boundaries between the low elasticity web portions 2550, 2560A and 2560B and the matrix fabric of the hose 2510 may be continuously knitted boundaries.

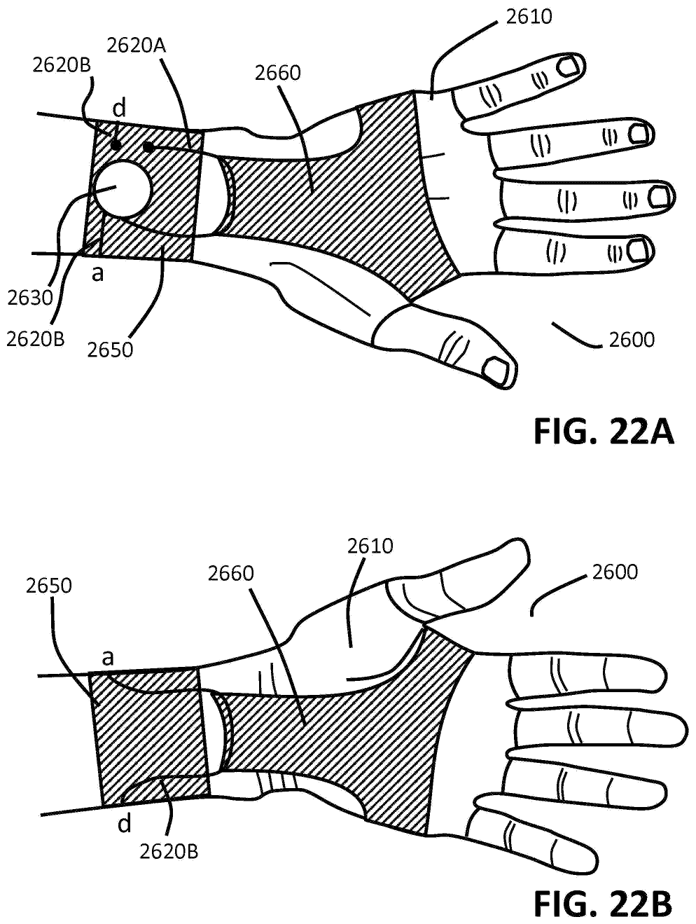

FIGS. 22A and 22B show a wrist brace garment 2600 comprising a fingerless glove 2610 of a knitted matrix fabric having a knitted low elasticity web portion 2660 disposed to brace the wrist joint when placed under tension. Tension is applied to the low elasticity web portion 2660 by means of two inextensible tension members 2620A and 2620B engaged with the low elasticity web portion 2660 and tensioned by tensioner 2630. Tensioner 2630 may optionally apply tension to inextensible tension members 2620A and 2620B via a tension evener (not shown). The tension evener may be, for example without limitation, any of the tension eveners described in published PCT application WO2021203191A1. Tensioner 2630 may be disposed on an anchor band 2650 disposed about the forearm above the wrist. Anchor band 2650 is employed as a stable base relative to which tension may be applied to low elasticity web portion 2660. Anchor band 2650 may also be knitted to be a low elasticity web portion. Tension members 2620A and 2620B may be guided by low elasticity web portion 2660 and 2650. Inextensible tension members 2620A and 2620B may be cables or strands arranged to move in knitted conduits within low elasticity web portions 2660 and 2650 and in the knitted matrix fabric of the glove 2610, as already described with reference to FIG. 14E. At least some of the boundaries between the low elasticity web portions 2650 and 2660A and the matrix fabric of the glove 2610 may be continuously knitted boundaries.

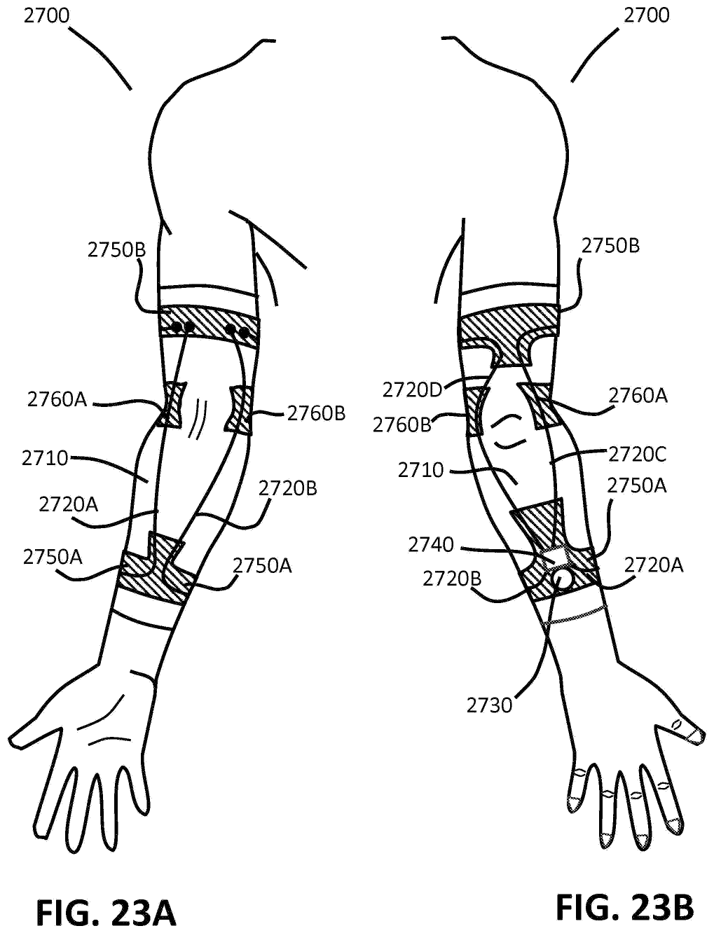

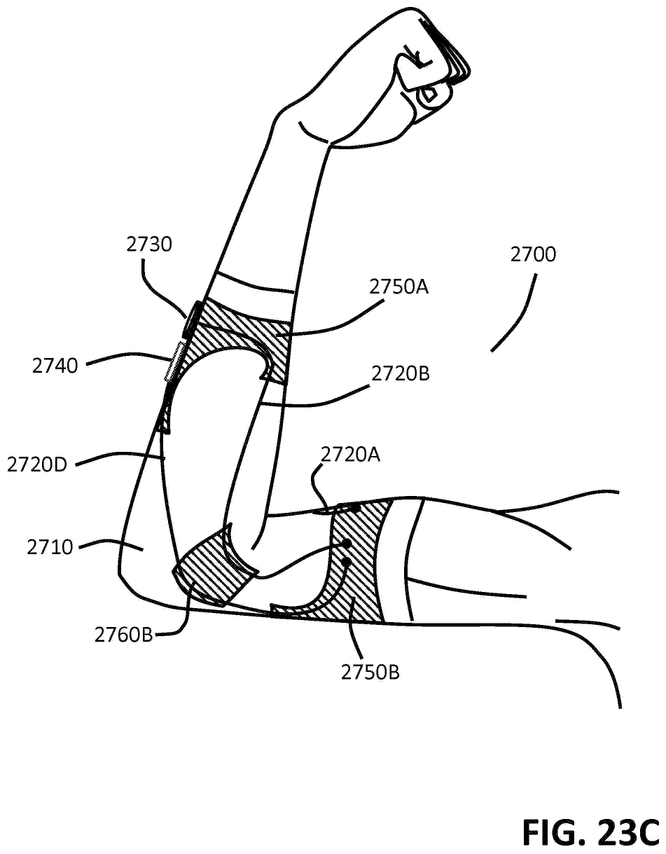

FIGS. 23A, 23B and 23C show an elbow brace garment 2700 comprising a hose 2710 of a knitted matrix fabric having knitted low elasticity web portions 2760A and 2760B disposed to brace the elbow joint when placed under tension. Tension is applied to the low elasticity web portions 2760A and 2760B by means of four inextensible tension members 2720A, 2720B, 2720C and 2720D. Inextensible tension members 2720A and 2720C are engaged with low elasticity web portion 2760A, and inextensible tension members 2720B and 2720D are engaged with low elasticity web portion 2760B.

Inextensible tension members 2720A, 2720B, 2720C and 2720D are all tensioned by tensioner 2730 via a tension evener 2740. The tension evener may be, for example without limitation, any of the tension eveners described in published PCT application WO2021203191A1. Tensioner 2730 may be disposed on an anchor band 2750A disposed about the forearm above the wrist. Anchor band 2750A is employed as a stable base relative to which tension may be applied to low elasticity web portions 2760A and 2760B. Anchor band 2750B is disposed about the upper arm and is employed as a stable base relative to which tension may be applied to low elasticity web portions 2760A and 2760B. Anchor bands 2750A and 2750B may also be knitted to be low elasticity web portions. Inextensible tension members 2720A, 2720B, 2720C and 2720D may be guided by low elasticity web portions 2760A, 2760B, 2750A and 2750B. Inextensible tension members 2720A, 2720B, 2720C and 2720D may be cables or strands arranged to move in knitted conduits within low elasticity web portions 2760A, 2760B, 2750A and 2750B and in the knitted matrix fabric of the hose 2710, as already described with reference to FIG. 14E. At least some of the boundaries between the low elasticity web portions 2760A, 2760B, 2750A and 2750B, and the matrix fabric of the hose 2710 may be continuously knitted boundaries.

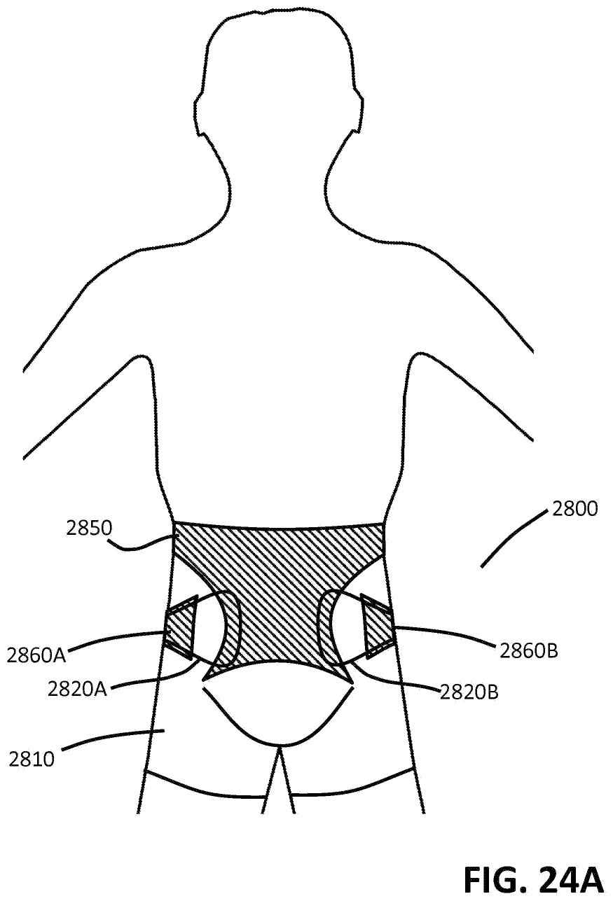

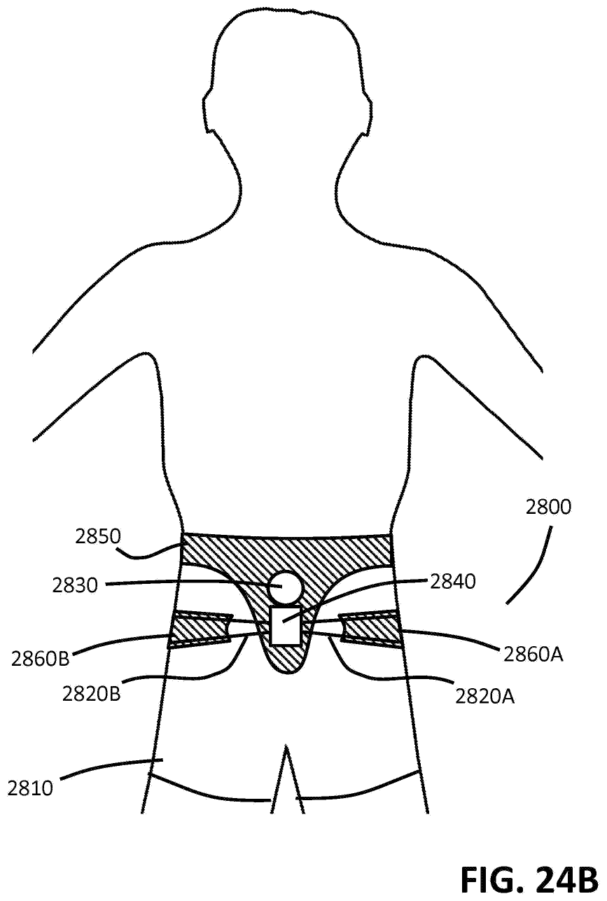

FIGS. 24A and 24B show a spinal brace garment 2800 comprising shorts 2810 of a knitted matrix fabric having a knitted low elasticity web portion 2850 disposed to brace the joints of the lower vertebrae when placed under tension. Tension is applied to the low elasticity web portion 2850 by means of two inextensible tension members 2820A and 2820B engaged with the low elasticity web portion 2850 and tensioned by tensioner 2830. Tensioner 2830 applies tension to inextensible tension members 2820A and 2820B via tension evener 2840. Tension evener 2840 may be, for example without limitation, any of the tension eveners described in published PCT application WO2021203191A1. Tensioner 2830 may be disposed on low elasticity web portion 2850 which also serves as anchor for garment 2800 as a whole. Tension members 2820A and 2820B may be guided by low elasticity web portions 2860A and 2860B. Inextensible tension members 2820A and 2820B may be cables or strands arranged to move in knitted conduits within low elasticity web portions 2850, 2860A and 2860B as well as in the knitted matrix fabric 2810, as already described with reference to FIG. 14E. At least some of the boundaries between the low elasticity web portions 2850, 2860A and 2860B and the matrix fabric 2810 may be continuously knitted boundaries.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A knitted brace garment configured for enveloping at least a first joint of a human user when the garment is worn by the user, the garment comprising:
an elastically extensible matrix fabric;
at least one low elasticity web portion having a first zonal fabric different from the matrix fabric; and
at least one segments of at least one tension member disposed to induce tension in the at least one low elasticity web portion,
wherein at least one boundary between the at least one low elasticity web portion and the matrix fabric is a continuously knitted boundary.

2. The brace garment of claim 1, wherein the at least one tension member is disposed along a path that spatially relates the at least one tension member to at least one natural ligament of the knee of the user, wherein at least one low elasticity web portion is disposed to apply pressure to one of a lateral side and a medial side of the knee of a wearer for bracing the knee.

3. The brace garment of claim 1, configured for enveloping a hip joint of a corresponding leg of a human user when the garment is worn by the user.

4. The brace garment of claim 1, comprising continuously knitted conduits for guiding the at least one tension member wherein the conduits are knitted into at least one of the matrix fabric and the at least one low elasticity web portion.

5. The brace garment of claim 4, wherein at least one of the conduits extends along a curved path arranged to circumnavigate in part a sensitive feature of the body of a user when the garment is worn by the user.

6. The brace garment of claim 4, wherein at least one of the conduits extends along a curved path arranged to circumnavigate in part a peroneal nerve of a leg of the user when the garment is worn by the user.

7. The brace garment of claim 1, comprising conduits disposed on top of the matrix fabric and the low elasticity web portions to guide the at least one tension member.

8. The brace garment of claim 1, wherein the at least one tension member is a closed loop.

9. The brace garment of claim 1, wherein the at least one tension member is anchored to the triceps surae of the user by a loop at least in part about the triceps surae.

10. The brace garment of claim 1, comprising tensioner on the garment arranged for adjusting a tension in the at least one tension member.

11. The brace garment of claim 1, wherein:
the at least one tension member comprises a plurality of strands; and
the garment comprises a tension evener arranged for evening tension among the plurality of strands.

12. The brace garment of claim 1, wherein the garment comprises a belt and the at least one tension member has a proximal end and a distal end, the proximal end of the at least one tension member being anchored to the belt and the distal end of the at least one tension member being disposed to be anchored by a triceps surae.

13. The brace garment of claim 1, wherein at least two segments of the at least one tension member are disposed to longitudinally extend along curved paths that traverse the at least one low elasticity web portion.

14. The brace garment of claim 13, wherein the curved paths curve in opposing directions to induce tension in the at least one low elasticity web portion.

15. The brace garment of claim 1, wherein at least two segments of the at least one tension member are disposed to longitudinally extend along a curved paths through the matrix fabric proximate two opposing boundaries between the at least one low elasticity web portion and the elastically extensible matrix.

16. The brace garment of claim 15, wherein the curved paths curve in opposing directions to induce tension in the at least one low elasticity web portion.

17. The brace garment of claim 1, wherein two segments of the at least one tension member extend along two corresponding curved paths traversing the at least one low elasticity web portion to induce tension between the two segments of the at least one tension member.

18. The brace garment of claim 1, wherein each of the at least one low elasticity web portion comprises at least one portion of the first zonal fabric knitted into the matrix fabric.

19. The brace garment of claim 18, wherein the at least one low elasticity web portion comprises at least one section of a second zonal fabric having a continuously knitted boundary with the at least one section of the first zonal fabric.

20. The brace garment of claim 1, comprising at least one low elasticity web portion disposed to partially envelop at least one limb of the user.

21. The garment of claim 1, wherein the first joint is one of an ankle, elbow, wrist, shoulder, hip, neck, spine, and one or more vertebrae of the human user.

22. The garment of claim 1, wherein the at least one low elasticity web portion includes an anchor band disposed to anchor the brace garment to a body of the human user.

23. A method for creating a brace garment for enveloping at least a first joint of a human user when the garment is worn by the user, the method comprising:
continuously knitting into an elastically extensible matrix fabric at least one low elasticity web portion having a zonal fabric different from the matrix fabric; and
continuously knitting into the elastically extensible matrix fabric conduits disposed for receiving inextensible tension members,
wherein the tension members when received in the conduits are disposed to induce tension in the at least one low elasticity web portion, and
wherein at least one boundary between the at least one low elasticity web portion and the matrix fabric is a continuously knitted boundary.

24. The method of claim 23, wherein the zonal fabric is continuously knitted from the same yarn as the matrix fabric.

25. The method of claim 23, wherein the zonal fabric is continuously knitted from a plurality of strands of the same yarn as the matrix fabric.

26. The method of claim 24, wherein the matrix fabric is knitted using a first stitch pattern and the zonal fabric is continuously knitted from the same yarn as the matrix fabric using a second stitch pattern different from the first stitch pattern.

27. The method of claim 23, wherein the zonal fabric is continuously knitted from one or more different yarns from the yarn of the matrix fabric.

28. The method of claim 23, wherein at least one of the different yarns is a melting yarn.

29. The method of claim 23, wherein the zonal fabric is continuously knitted from the same yarn as the as the matrix fabric and from one or more yarns that differ from the yarn of the matrix fabric.

30. The method of claim 23, wherein the matrix fabric is knitted using a first stitch pattern and the zonal fabric is continuously knitted using a second stitch pattern different from the first stitch pattern.

31. The method of claim 23, wherein the conduits are continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

32. The method of claim 23, wherein the zonal fabric is continuously knitted using at least one of a different stitch pattern from a stitch pattern of the matrix fabric, a different yarn from the yarn used for knitting the matrix fabric, and a different number of strands of yarn than the number of strands of yarn used for knitting the matrix fabric.

33. The method of claim 23, wherein the first joint is one of an ankle, elbow, wrist, shoulder, hip, neck, spine, and one or more vertebrae of the human user.
